SEQUENCE LISTING (31 sequences)

SEQ ID NO: 1 - protein, 7 aa, Campylobacter jejuni
KIDLNNT

SEQ ID NO: 2 - DNA, 264 bp, Campylobacter jejuni
ctcgagttcataaaaaatttcaagcaacatgaaaaaattaagatagatcttaataatacaaagatagatcttaataatacaaagatagatcttaataatacaaagatagatcttaataatacaaagatagatcttaataatacaaagatagatcttaataatacaaagatagatcttaataatacaaagatagatcttaataatacaaagatagatcttaataatacaaagatagaattatcgcaattaaaaaaagagctcgag

SEQ ID NO: 3 - DNA, 27 bp, Artificial Sequence
  primer
atggccatggataataaaaaatcagcc

SEQ ID NO: 4 - DNA, 35 bp, Artificial Sequence
  primer
tttctcgagtttgcttcctttggcatcatttatcg

SEQ ID NO: 5 - DNA, 28 bp, Artificial Sequence
  primer
aaactcgagttcataaaaaatttcaagc

SEQ ID NO: 6 - DNA, 27 bp, Artificial Sequence
  primer
atatctcgagctctttttttaattgcg

SEQ ID NO: 7 - protein, 468 aa, Artificial Sequence
  Camplyobacter fetus fetus/Campylobacter jejuni
MKYLLPTAAAGLLLLAAQPAMAMDNKKSAAQQQIPPMPVTVMQAKMGDIPIVLSFNGQTVSDMDVVLKAKVAGTIEKQFFKAGASVKEGDKLYQIDEAKYRAAYDSAFANLQVSQANLKNAESDFDRAKKLQEKSAISQKEYDAALAAYESAKASVLASKANVQTAKIDLDYSTVTAPFSGVLGDTLKDVGSYVSSSDADLVRLTKLNPIFVKFGISDVDKLNIAQKTRTKEWEQLNSLVTLSMQNGDYNGTLTFIDNVIDEGSGTVNAKAMFENNSTQIRPGIFASVNVYGFYQKNGFKIPQVAILQDLASPFVYTLKDGKVAKNPIKIVSQDSTSAVISSGINDGDLIIMDNFKKINIGQPVQAINDAKGSKLEFIKNFKQHEKIKIDLNNTKIDLNNTKIDLNNTKIDLNNTKIDLNNTKIDLNNTKIDLNNTKIDLNNTKIDLNNTKIELSQLKKELEHHHHHH

SEQ ID NO: 8 - protein, 469 aa, Artificial Sequence
  Camplyobacter fetus fetus/Campylobacter jejuni
MKYLLPTAAAGLLLLAAQPAMAMVIIKNFKQHEKIKIDLNNTKIDLNNTKIDLNNTKIDLNNTKIDLNNTKIDLNNTKIDLNNTKIDLNNTKIDLNNTKIELSQLKKEPMDNKKSAAQQQIPPMPVTVMQAKMGDIPIVLSFNGQTVSDMDVVLKAKVAGTIEKQFFKAGASVKEGDKLYQIDEAKYRAAYDSAFANLQVSQANLKNAESDFDRAKKLQEKSAISQKEYDAALAAYESAKASVLASKANVQTAKIDLDYSTVTAPFSGVLGDTLKDVGSYVSSSDADLVRLTKLNPIFVKFGISDVDKLNIAQKTRTKEWEQLNSLVTLSMQNGDYNGTLTFIDNVIDEGSGTVNAKAMFENNSTQIRPGIFASVNVYGFYQKNGFKIPQVAILQDLASPFVYTLKDGKVAKNPIKIVSQDSTSAVISSGINDGDLIIMDNFKKINIGQPVQAINDAKGSKLEHHHHHH

SEQ ID NO: 9 - protein, 467 aa, Artificial Sequence
  Camplyobacter fetus fetus/Campylobacter jejuni
MKYLLPTAAAGLLLLAAQPAMAMDNKKSAAQQQIPPMPVTVMQAKMGDIPIVLSFNGTVSDMDVVLKAKVAGTIEKQFFKAGASVKEGDKLYQIDEAKYRAAYDSAFANLQVSQANLKNAESDFDRAKKLQEKSAISQKEYDAALAAYESAKASFIKNFKQHEKIKIDLNNTKIDLNNTKIDLNNTKIDLNNTKIDLNNTKIDLNNTKIDLNNTKIDLNNTKIDLNNTKIELSQLKKEASVLASKANVQTAKIDLDYSTVTAPFSGVLGDTLKDVGSYVSSSDADLVRLTKLNPIFVKFGISDVDKLNIAQKTRTKEWEQLNSLVTLSMQNGDYNGTLTFIDNVIDEGSGTVNAKAMFENNSTQIRPGIFASVNVYGFYQKNGFKIPQVAILQDLASPFVYTLKDGKVAKNPIKIVSQDSTSAVISSGINDGDLIIMDNFKKINIGQPVQAINDAKGSKLEHHHHHH

SEQ ID NO: 10 - protein, 63 aa, Campylobacter jejuni
KIDLNNTKIDLNNTKIDLNNTKIDLNNTKIDLNNTKIDLNNTKIDLNNTKIDLNNTKIDLNNT

SEQ ID NO: 11 - DNA, 33 bp, Artificial Sequence
  primer
aatatataccatgggttgcactcgggcaattcg

SEQ ID NO: 12 - DNA, 35 bp, Artificial Sequence
  primer
atatatctcgagcgttaccctgcggatgttttcgg

SEQ ID NO: 13 - DNA, 49 bp, Artificial Sequence
  primer
tatatggccatggcaacaccaattccattttcttattctaaaaatctgg

SEQ ID NO: 14 - DNA, 49 bp, Artificial Sequence
  primer
ttaaactcgagatcatttgtccatccttcatctgtaggtacaaagtacc

SEQ ID NO: 15 - DNA, 36 bp, Artificial Sequence
  primer
atatatatccatggctgctgatgatgttgttgattc

SEQ ID NO: 16 - DNA, 37 bp, Artificial Sequence
  primer
atatactcgagtcgcctgacacgatttcctgcacagg

SEQ ID NO: 17 - DNA, 44 bp, Artificial Sequence
  primer
gcaattaaaaaaagagatcgagcaccaccaccaccaccactgag

SEQ ID NO: 18 - DNA, 44 bp, Artificial Sequence
  primer
ctcagtggtggtggtggtggtgctcgatctctttttttaattgc

SEQ ID NO: 19 - DNA, 45 bp, Artificial Sequence
  primer
gctgcccagccggcgatggcgatggttatcataaaaaatttcaag

SEQ ID NO: 20 - DNA, 45 bp, Artificial Sequence
  primer
cttgaaattttttatgataaccatcgccatcgccggctgggcagc

SEQ ID NO: 21 - DNA, 46 bp, Artificial Sequence
  primer
atatctcgaggcggccgcaagctgaggagactgtgaccatgactcc

SEQ ID NO: 22 - DNA, 34 bp, Artificial Sequence
  primer
aattctgcagtaattttgtttaactttaagaagg

SEQ ID NO: 23 - DNA, 38 bp, Artificial Sequence
  primer
atatatatccatgggaggtggcggatcagaagtgaagc

SEQ ID NO: 24 - DNA, 31 bp, Artificial Sequence
  primer
atatggtaccttagcagccggatctcagtgg

SEQ ID NO: 25 - DNA, 28 bp, Artificial Sequence
  primer
aaaataaatataggacaaccggttcagg

SEQ ID NO: 26 - DNA, 29 bp, Artificial Sequence
  primer
tatctcgagtcactctttttttaattgcg

SEQ ID NO: 27 - protein, 506 aa, Artificial Sequence
  Campylobacter jejuni/Mus musculus
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFGSSRVEFIKNFKQHEKIKIDLNNTKIDLNNTKIDLNNTKIDLNNTKIDLNNTKIDLNNTKIDLNNTKIDLNNTKIDLNNTKIELSQLKKE

SEQ ID NO: 28 - DNA, 28 bp, Artificial Sequence
  primer
aaaataaatataggacaaccggttcagg

SEQ ID NO: 29 - DNA, 34 bp, Artificial Sequence
  primer
aatttaagctttgttagcagccggatctcagtgg

SEQ ID NO: 30 - protein, 514 aa, Artificial Sequence
  Campylobacter jejuni/Mus musculus
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFGSSRVEFIKNFKQHEKIKIDLNNTKIDLNNTKIDLNNTKIDLNNTKIDLNNTKIDLNNTKIDLNNTKIDLNNTKIDLNNTKIELSQLKKEIEHHHHHH

SEQ ID NO: 31 - protein, 150 aa, Campylobacter jejuni
MQRFKKWFLSIIKNFKQHEKIKIDLNNTKIDLNNTKIDLNNTKIDLNNTKIDLNNTKIDLNNTKIDLNNTKIDLNNTKIDLNNTKIELSQLKKEHYKVLDFHLRKITPQAFLEIVEIHLAESCNLNCFGCNHFSQIAEKEFPDIEIFKKD

(19) United States
(12) Patent Application Publication
(10) Pub. No.: US 2013/0266604 A1
(43) Pub. Date: Oct. 10, 2013

(54) PEPTIDE CONTAINING MULTIPLE N-LINKED GLYCOSYLATION SEQUONS

(71) Applicants: Christine Szymanski, Edmonton (CA); Harald Nothaft, Edmonton (CA)

(72) Inventors: Christine Szymanski, Edmonton (CA); Harald Nothaft, Edmonton (CA)

(73) Assignee: The Governors of the University of Alberta a non-profit educational institution

(21) Appl. No.: 13/819,645

(22) PCT Filed: Sep. 2, 2011
(86) PCT No.: PCT/CA2011/050535

§ 371 Date: May 20, 2013

Related U.S. Application Data
(60) Provisional application No. 61/379,896, filed on Sep. 3, 2010.

(51) Int. Cl. C07K 7/06

(52) U.S. Cl. CPC C07K 7/06
USPC 424/190.1; 530/327; 530/326; 530/324; 530/350; 530/395; 530/322; 530/387.3; 435/69.1; 435/7.1; 435/69.6; 435/69.3

(57) ABSTRACT

Described herein is a peptide, referred to as a GlycoTag peptide, that includes repeats of an N-linked glycosylation sequon that is naturally present in C. jejuni. The GlycoTag peptide can be in a purified and isolated form following expression of a construct encoding the GlycoTag peptide or it is, in whole or in part, a synthetic peptide. The GlycoTag peptide is typically used with glycans attached thereto. The GlycoTag peptide can be used alone or in combination with different proteins or peptides and can be incorporated into a chimeric protein or peptide for use in the development of antigen/antibody delivery systems, the preparation of vaccines, pharmaceuticals and diagnostic tests, and research into therapies for countering Campylobacter and other infections.

### CROSS-REFERENCE TO RELATED APPLICATION

This application claims the benefit and priority to U.S. provisional patent application No. 61/379,896, filed Sep. 3, 2010, which is incorporated herein in its entirety as though set forth explicitly herein.

### FIELD OF THE INVENTION

The invention relates to campylobacter-derived peptides which contain multiple sequential sequons for N-linked glycosylation.

### BACKGROUND

Asparagine (N-linked) protein glycosylation was once believed to exist in only eukaryotes and archaea until its discovery in the e-Proteobacterium Campylobacter jejuni (Szymanski C M et al., 1999: Evidence for a system of general protein glycosylation in Campylobacter jejuni. Mol Micro, 32 (5): 1022-1030; Wacker M et al., 2002: N-linked glycosylation in Campylobacter jejuni and its functional transfer into E. coli. Science, 298 (5599):1790-1793; Young N M et al., 2002: Structure of the N-linked glycan present on multiple glycoproteins in the Gram-negative bacterium, Campylobacter jejuni. J Biol Chem, 277 (45):42530-42539). In eukaryotes, N-linked glycosylation is an essential process and is catalyzed by an oligosaccharyltransferase (OST) complex consisting of 9 proteins with STT3 being the catalytic subunit (for a review see: Yan A & Lennarz W J., 2005: Unraveling the mechanism of protein N-linked glycosylation. J Biol Chem, 280 (5):3121-3124). In C. jeuni, N-linked glycosylation occurs in the periplasm and PglB, the STT3 orthologous protein, is the key enzyme responsible for the transfer of oligosaccharides to acceptor peptides and proteins. The presence of a consensus sequence (an N-linked glycosylation sequon) within the secreted acceptor peptides is required for recognition by PglB. This sequon consists of D/E-X₁—N—X₂—S/T, where X₁ and X₂ can be any amino acid except proline (Kowarik M et al., 2006: Definition of the bacterial N-glycosylation site consensus sequence. EMBO J, 25 (9):1957-1966).

The current model for assembly of the C. jejuni N-linked glycan is shown in FIG. 1 (Szymanski C M et al., 2003: Campylobacter—a tale of two protein glycosylation systems. Trends Microbiol, 5 (11): 233-238). The bactoprenyl-pyrophosphate-linked heptasaccharide is assembled in the cytosol by the addition of nucleotide-activated sugars (Szymanski C M et al., 2003: Campylobacter—a tale of two protein glycosylation systems. Trends Microbiol, 5 (11): 233-238; Szymanski C M et al., 2003: Detection of conserved N-linked glycans and phase-variable lipooligosaccharides and capsules from campylobacter cells by mass spectrometry and high resolution magic angle spinning NMR spectroscopy. J Biol Chem, 278 (27): 24509-24520). The complete heptasaccharide is translocated across the inner membrane to the periplasm by the ATP-Binding Cassette (ABC) transporter PglK (Alaimo C et al., 2006: Two distinct but interchangeable mechanisms for flipping of lipid-linked oligosaccharides. EMBO J, 25 (5):967-976). The oligosaccharide is then transferred to an N-linked glycosylation sequon by PglB or released into the periplasmic space as free oligosaccharide (fOS) (Nothaft et al., 2009: Study of free oligosaccharides derived from the bacterial N-glycosylation pathway. PNAS, 106 (35): 15019-15024). (Young N M et al., 2002: Structure of the N-linked glycan present on multiple glycoproteins in the Gram-negative bacterium, Campylobacter jejuni. J Biol Chem, 277 (45): 42530-42539; Wacker M et al., 2002: N-linked glycosylation in Campylobacter jejuni and its functional transfer into E. coli. Science, 298 (5599):1790-1793).

It has been demonstrated that the N-linked protein glycosylation machinery of C. jejuni can be functionally transferred into Escherichia coli (Wacker M et al., 2002: N-linked glycosylation in Campylobacter jejuni and its functional transfer into E. coli. Science, 298 (5599):1790-1793). This has lead to a surge of activity to exploit this pathway for glycoprotein engineering and has opened up the possibility of engineering glycan structures for various purposes, which include but are not limited to, furthering research of N-linked glycosylated proteins and N-linked glycosylation, and therapeutic applications including the production of therapeutically useful antibodies and vaccines.

However, N-linked glycoprotein engineering using the C. jejuni glycosylation machinery in E. coli has several disadvantages and limitations. One of the most important limitations to recombinant glycoprotein production is the number of N-linked glycosylation sequons present within a natural glycoprotein. As one of skill in the art will appreciate, currently available molecular biology techniques allow for the insertion of additional N-linked glycosylation sequons in naturally-glycosylated proteins and even the insertion of N-linked glycosylation sequons in proteins that are not normally glycosylated. However, insertion of these N-linked glycosylation sites follows a mostly trial-and-error approach. Without significant knowledge of a protein's three-dimensional structure, some N-linked glycosylation sites may be inserted in regions of the protein that will not be accessible to the protein glycosylation machinery (Kowarik M et al., 2006: N-linked glycosylation of folded proteins by the bacterial oligosaccharyltransferase. Science, 314 (5802): 1148-1150; Kowarik M et al., 2006: Definition of the bacterial N-linked glycosylation site consensus sequence. EMBO J, 25 (9):1957-1966). Moreover, lack of information on the three-dimensional structure of a protein may prevent the insertion of sequential N-linked glycosylation sequons. The N-linked glycosylation sequon identified by Kowarik M et al. (2006) was identified as forming a segment within a protein, but wherein only a single such sequon was identified within a protein, or the sequons were widely spaced within the protein. Without structural information of proteins, it is very difficult to introduce multiple N-linked glycosylation sequons or sequential sequons in a protein and ensure efficient glycosylation in a recombinant host.

Furthermore, the engineering of N-linked glycosylation sequons in a protein or peptide is a time-consuming and often costly process. This can be particularly problematic for the development of therapeutic antibodies and/or vaccines.

Consequently, the need has arisen for peptides and proteins that contain multiple N-linked glycosylation sequons, that can be efficiently and recombinantly glycosylated, while avoiding some of the problems listed above.

This background information is provided for the purpose of making known information believed by the applicant to be of possible relevance to the present invention. No admission is necessarily intended, nor should be construed, that any of the preceding information constitutes prior art against the present invention.

### SUMMARY

The object of the present application is to provide a peptide containing multiple N-linked glycosylation sequons, and methods of preparation and use thereof. In accordance with a broad aspect, there is provided a peptide comprising at least two copies of the amino acid sequence set forth in SEQ ID NO: 1 (this peptide is referred to herein as a “GlycoTag” peptide). In one embodiment, the GlycoTag peptide comprises nine copies of the amino acid sequence set forth in SEQ ID NO: 1. The copies of the amino acid sequence set forth in SEQ ID NO: 1 can be contiguous or can be separated by no more than 6 amino acids. The GlycoTag peptide can further comprise a signal peptide that facilitates periplasmic secretion.

In accordance with another broad aspect, there is provided a chimeric protein comprising at least one GlycoTag peptide and a second peptide or protein. The GlycoTag comprises at least two copies of the amino acid sequence set forth in SEQ ID NO: 1, which can be contiguous or separated by no more than 6 amino acids. In one embodiment, the second peptide or protein comprises a protein derived from, or native to, a Campylobacter. In one embodiment, the second protein is AcrA. In one embodiment, the second protein or peptide is selected from the group consisting of a toxin, a toxoid, an antibody and a periplasmic protein. The toxin or toxoid may be derived from the group selected from Bordetella pertussis, Clostridium tetani, and Corynebacterium diphtheriae. In one embodiment, the antibody selected as the second protein or peptide targets a dendritic cell.

In accordance with another broad aspect, there is provided a method for producing a chimeric protein comprising multiple glycosylation sites, the method comprising the steps of engineering a chimeric gene comprising a nucleic acid sequence encoding a GlycoTag peptide operably linked, directly or via a linker, to a nucleic acid sequence encoding a second peptide or protein, for example a protein or peptide that is capable of targeting the chimeric protein to the periplasmic space, and expressing the resulting chimeric gene in a host. The host can be E. coli.

In one embodiment, the second peptide or protein comprises a toxin or toxoid, an antibody or a periplasmic protein. In one embodiment, the toxin or toxoid is derived from Bordetella pertussis, Clostridium tetani, or Corynebacterium diphtheriae. In one embodiment, the second protein comprises an antibody and the chimeric protein is for use in an adjuvant-free composition for treating a Campylobacter infection. In one embodiment, the antibody targets a dendritic cell. In one embodiment, the second protein or peptide is derived from, or native to, a Campylobacter. In another embodiment, the second protein is AcrA.

In one embodiment, the method for producing a chimeric protein comprises the further step of glycosylating the chimeric peptide or protein with a plurality of N-linked glycans. In one embodiment, the N-linked glycans are derived from C. jejuni.

The GlycoTag peptide described herein can be used for many different purposes. In one embodiment, the GlycoTag peptide can be used to display N-glycans of eukaryotic, bacterial and/or archaeal origin. In another embodiment, it can be used to display O-glycans. In one embodiment, the GlycoTag peptide when glycosylated with specific glycans can be used as a glycan carrier for vaccination against zoonotic human pathogens selected from the group consisting of Campylobacter upsaliensis in cats and dogs, Campylobacter coli in pigs and Campylobacter jejuni in chickens. In another embodiment, it can be used as a glycan carrier for vaccination against Travelers' Diarrhea caused by Campylobacter jejuni. In another embodiment, it can be used as a glycan carrier for vaccination of cows and sheep to prevent infertility and abortions caused by Campylobacter fetus venerealis and Campylobacter fetus fetus. In yet another embodiment, it can be used in a diagnostic method in which it is used to display bacterial glycans and to then determine the presence or absence, or to quantify, anti-sera in a sample obtained from a subject suspected of having a bacterial infection.

In another embodiment, the GlycoTag peptide of the present invention can be used as an adjuvant in vaccine preparation. It can also be used in combination with a second protein or peptide for the preparation of a vaccine or pharmaceutical. The second peptide or protein can comprise a protein derived from or native to a Campylobacter. In one embodiment, the second protein is AcrA. In one embodiment, the second protein or peptide is selected from the group consisting of a toxin, toxoid, an antibody and a periplasmic protein. The toxin or toxoid can be derived from Bordetella pertussis, Clostridium tetani, or Corynebacterium diphtheriae. In one embodiment, the antibody selected as the second protein targets a dendritic cell.

### BRIEF DESCRIPTION OF THE DRAWINGS

The present invention, both as to its organization and manner of operation, may best be understood by reference to the following description, and the accompanying drawings of various embodiments wherein like numerals are used throughout the several views, and in which:

FIG. 1 is a schematic diagram of the process of N-linked glycosylation in C. jejuni as determined in the prior art.

FIG. 2A is the graphical output from a bioinformatic analysis of a segment of the Cj1433c protein using SignalP-NN. The first 70 amino acids of the 1433c protein were analyzed with the SignalP program (version 3.0) (http://www.cbs.dtu.dk/services/SignalP/) to evaluate the possibility of Cj1433c being a periplasmic protein. The algorithm clearly shows the absence of a signal peptide sequence necessary for secretion into the periplasmic space of C. jejuni, which is a prerequisite for N-linked protein glycosylation.

FIG. 2B is the graphical output from a bioinformatic analysis of a segment of the Cj1433c protein using SignalP-HMM. The first 70 amino acids of the 1433c protein were analyzed with the SignalP program (version 3.0) (http://www.cbs.dtu.dk/services/SignalP/) to evaluate the possibility of Cj1433c being a periplasmic protein. The algorithm clearly shows the absence of a signal peptide sequence necessary for secretion into the periplasmic space of C. jejuni, which is a prerequisite for N-linked protein glycosylation.

FIG. 3 shows the sequence of the first 150 amino acids of the C. jejuni 1433c protein (SEQ ID NO: 31). The sequence containing the GlycoTag peptide (nine repeats of SEQ ID NO: 1) is located between ** and **. The bacterial N-glycosylation sequon (DLNNT) is underlined.

FIG. 4 shows the composition of construct nos. 1, 2 and 3, where the GlycoTag was fused to an AcrA construct. For constructs 1, 2 and 3, AcrA, an N-glycoprotein of Campylobacter fetus fetus (Cff), was used (AcrA^{Cff}). The GlycoTag peptide was inserted at the N-terminus in construct 1 (to yield GlycoTag-AcrA^{Cff}-His fusion protein), at the C-terminus in construct 2 (to yield AcrA^{Cff}-GlycoTag-His fusion protein), and in between the lipoyl- and the α-hairpin domain of the AcrA protein in construct 3 (to yield N-AcrA^{Cff}-GlycoTag-C-AcrA^{Cff}-His fusion protein). “ss” stands for pectate lyaseB leader sequence, while “His6” stands for a hexa-histidine tag.

FIG. 5 depicts a photograph of an SDS-PAGE gel demonstrating the expression of the first generation of GlycoTag-AcrA^{Cff} fusion proteins produced using the constructs described in FIG. 4. The pectate lyaseB leader sequence (ss) was used to direct the protein to the periplasm. Expression of the fusion proteins in E. coli BL21 grown at 28° C. was followed by western blotting (anti-His) after 10% SDS-PAGE of cells after induction with 0.1 mM IPTG for 18 h. Only the C-terminal fusion construct (construct 2, lane 2) resulted in a protein with the expected size of 55 kDa.

FIG. 6A is the amino acid sequence of the AcrA^{Cff}-GlycoTag-His fusion protein as per construct 1 shown in FIG. 4. The pET22b-derived pelB leader (ss) and hexa-histidine amino acid sequences are in italics, the N-linked glycosylation sequons are in bold and the GlycoTag peptide is underlined.

FIG. 6B is the amino acid sequence of the GlycoTag-AcrA^{Cff}-His fusion protein as per construct 2 shown in FIG. 4. The pET22b-derived pelB leader (ss) and hexa-histidine amino acid sequences are in italics, the N-linked glycosylation sequons are in bold and the GlycoTag peptide is underlined.

FIG. 6C is the amino acid sequence of the interdomain N-AcrA^{Cff}-GlycoTag-C-AcrA^{Cff}-His fusion protein as per construct 3 shown in FIG. 4. The pET22b-derived pelB leader (ss) and hexa-histidine amino acid sequences are in italics, the N-linked glycosylation sequons are in bold and the GlycoTag peptide is underlined.

FIG. 7A depicts a photograph of an electrophoretogram showing purification of the soluble, periplasmically-expressed AcrA^{Cff}-GlycoTag-His fusion protein (construct 2) in the presence of the pgl machinery of C. jejuni. Ni-NTA affinity chromatography was used for purification. Lane 1 is a sample of crude cell lysate; lane 2 is a sample of column flow-through; and lane 3 is a sample of the eluate obtained with 250 mM imidazole.

FIG. 7B depicts a photograph of an immunoblot of His-tagged proteins produced by western blotting with anti-His antibodies. Lane 1 is a sample of the AcrA^{Cff} protein and lane 2 is a sample of the AcrA^{Cff}-GlycoTag-His fusion protein.

FIG. 7C depicts a photograph of an immunoblot of His-tagged proteins produced by western blotting with C. jejuni N-glycan-specific antiserum. Lane 1 is a sample of the AcrA^{Cff} protein and lane 2 is a sample of the AcrA^{Cff}-GlycoTag-His fusion protein.

FIG. 7D is an expanded view of lane 2 of FIG. 7C, showing the N-glycosylated fusion protein with up to 11 heptasaccharides.

FIG. 8A is a schematic diagram of a construct used to produce a toxoid-GlycoTag fusion protein in the heterologous E. coli protein glycosylation system (toxC_{Cd}-GlycoTag fusion protein). The GlycoTag was fused to the C-terminus of the N-terminal portion (nt 79 to nt 654) of the toxC gene of Corynebacterium diphtheriae NCTC 13129. “ss” stands for pectate lyaseB leader sequence, while “His6” stands for a hexa-histidine tag.

FIG. 8B is a schematic diagram of a construct used to produce a dendritic cell targeting single chain antibody-GlycoTag fusion protein (scFv-GlycoTag fusion protein) in the heterologous E. coli protein glycosylation system. The GlycoTag was fused to the C-terminus of a scFv that recognizes the DEC-205 receptor on mouse dendritic cells. “ss” stands for pectate lyaseB leader sequence, while “His6” stands for a hexa-histidine tag.

FIG. 8C depicts a photograph of an electrophoretogram of a 10% SDS-PAGE gel showing expression of the toxC_{Cd}-GlycoTag fusion protein in the heterologous E. coli protein glycosylation system. The fusion protein was purified using Ni-NTA affinity chromatography. Lane 1 is a sample of whole cell lysate of cells after induction of protein expression with 0.1 mM IPTG for 18 h; lane 2 is a sample of flowthrough from the Ni-NTA affinity chromatography; and lane 3 is a sample of protein eluted with 250 mM imidazole.

FIG. 8D depicts a photograph of an electrophoretogram showing immunodetection of the glycosylation of the toxC_{Cd}-GlycoTag fusion protein with Campylobacter jejuni N-glycan specific hR6 antiserum in E. coli (pgl+), lane 1, or E. coli (pgl−), lane 2.

FIG. 8E depicts a photograph of an electrophoretogram of a 10% SDS-PAGE gel showing expression of the scFv-GlycoTag fusion protein in the heterologous E. coli protein glycosylation system. The fusion protein was purified using Ni-NTA affinity chromatography. Lane 1 is a sample of whole cell lysate of cells after induction of protein expression with 0.1 mM IPTG for 18 h; lane 2 is a sample of flowthrough from the Ni-NTA affinity chromatography; and lane 3 is a sample of protein eluted with 250 mM imidazole.

FIG. 8F depicts a photograph of an electrophoretogram showing immunodetection of the glycosylation of the scFv-GlycoTag fusion protein with hR6 antiserum in E. coli (pgl+), lane 1, or E. coli (pgl−), lane 2.

FIG. 9A is a schematic diagram of a construct used to produce a toxoid-(GlycoTag)₂ fusion protein in the heterologous E. coli protein glycosylation system (toxC_{Cd}-(GlycoTag)₂ fusion protein). Two sequential copies of the GlycoTag peptide were fused to the C-terminus of the N-terminal portion (nt 79 to nt 654) of the toxC gene of Corynebacterium diphtheriae NCTC 1312. “ss” stands for pectate lyaseB leader sequence, while “His6” stands for a hexa-histidine tag.

FIG. 9B illustrates detection of the toxC_{Cd}-GlycoTag and toxC_{Cd}-(GlycoTag)₂ fusion proteins expressed and glycosylated with the C. jejuni heptasaccharide. A western blot with hR6 antiserum after 12% SDS-PAGE of Ni-NTA affinity-purified (as described above in FIG. 8) toxC_{Cd}-GlycoTag protein (lane 2) and toxC_{Cd}-(GlycoTag)₂ (lane 3) fusion proteins. Protein expression in E. coli BL21 in the presence of the C. jejuni protein glycosylation operon on plasmid pACYC was induced with 0.1 mM IPTG for 18 h. For clarity, individual glycoprotein-specific signals are marked by a line on the side of the corresponding signal. The sizes of molecular weight markers in kDa (lane 1) are indicated on the left.

FIG. 10 is a bar chart summarizing the N-glycan-specific antibody response. ELISA Plates (Nune 96-well Maxisorb) were coated overnight with monovalent LipidA-N-glycan conjugate (prepared according to Manoharan R et al., 1990: UV resonance Raman spectra of bacteria, bacterial spores, protoplasts, and calcium dipicolinate. J. Microbiol. Methods 11 (1): 1-15) from an E. coli O-antigen polymerase mutant strain that expresses the protein glycosylation gene cluster of C. jejuni (Wacker M et al., 2002: N-linked glycosylation in Campylobacter jejuni and its functional transfer into E. coli. Science, 298 (5599):1790-1793)). Plates were washed 3-times with PBST (0.1% Tween 20 in PBS, pH 7.3) followed by a blocking step with 2% skim milk for 3 h at RT. Plates were washed once with PBST and the 1:50 diluted (in PBST) mouse sera were added and incubated for 2 h at RT. Plates were then washed with PBST and incubated with alkaline-phosphatase labeled anti-mouse antibody (Santa Cruz, 1:1000 dilution in PBST). After 1 h at room temperature, the plates were washed 3-times with PBST, PNPP was added to each well, and the OD₄₀₅ nm and the OD₅₇₀ nm (background) was taken after 30 min using a microplate reader. Each bar represents the background subtracted values for the indicated antigen group; standard deviations that represent 2 experimental and 2 technical replicates are indicated by error bars.

FIG. 11A is a bar chart summarizing the N-glycan-specific antibody response from day 21. ELISA plates were coated as described (FIG. 10). Analysis of the N-glycan-specific immune response using diluted mouse sera (1:100 in PBST) was done as described (in FIG. 10).

FIG. 11B is a bar chart summarizing the N-glycan-specific antibody response from day 35. ELISA plates were coated as described (FIG. 10). Analysis of the N-glycan-specific immune response using diluted mouse sera (1:100 in PBST) was done as described (in FIG. 10).

FIG. 12A represents the colony counts (cfu) obtained after colonization of 1-day-old chickens by Campylobacter jejuni in the groups as outlined in table 3. The bar represents the median level of colonization for each group.

FIG. 12B illustrates the IgG (IgY) humoral response in group 4 and group 5 chicken sera after immunoblotting against Proteinase K digested E. coli extracts producing monovalent Lipid A-linked N-glycan of C. jejuni (+) or LipidA core (−) that were separated on 16% DOC gels and probed with sera from different chicken (final bleed, [group-chicken number as indicated]). The molecular weight of the N-glycan-specific signals when compared to the positive control (positive [+ve] control) probed with N-glycan-specific antiserum, hR6, are indicated by an arrow. No N-glycan-specific signal was obtained with pooled sera obtained from the final bleed of group 1 chickens (negative [-ve]control).

FIG. 13 represents the colony counts (cfu) obtained after colonization of 1-day-old chickens by Campylobacter jejuni in the groups as outlined in table 4. The bar represents the median level of colonization for each group.

FIG. 14 is a schematic diagram of a construct used to produce a Maltose-binding-protein-factor Xa-GlycoTag-hexa-histidine fusion protein in the heterologous E. coli protein glycosylation system (MalE-GT-His). The GlycoTag is fused to the C-terminus of MalE that can be purified using amylose-agarose affinity chromatography. “Ptac” stands for IPTG-inducible promoter, “MalE” stands for Maltose-binding-protein, “Xa-site” stands for factor Xa protease recognition sequence “ss” stands for pectate lyaseB leader sequence, while “His6” stands for a hexa-histidine tag.

FIG. 15 illustrates detection of the GlycoTag peptide and detection of the GlycoTag peptide glycosylated with the C. jejuni heptasaccharide. Western blots with His-Tag-specific and Campylobacter jejuni N-glycan specific hR6 antiserum after 15% SDS-PAGE of Ni-NTA affinity-purified (as described above in FIG. 8) GlycoTag after digestion of amylose-agarose-affinity purified MalE-GlycoTag fusion protein with factor Xa protease (lane 1, His-Tag specific antiserum; lane 3, hR6 antiserum) and of Ni-NTA affinity-purified (as described above in FIG. 8) glycosylated GlycoTag expressed in the presence of the protein glycosylation machinery of C. jejuni after digestion of amylose-agarose-affinity MalE-GlycoTag fusion protein with factor Xa protease (lane 2, His-Tag specific antiserum; lane 4, hR6 antiserum) is shown. Protein expression in E. coli DH5α in the presence or the absence of the C. jejuni protein glycosylation operon on plasmid pACYC was induced with 0.1 mM IPTG for 18 h. For clarity, individual glycoprotein-specific signals are marked by a line on the side of the corresponding signal. The sizes of molecular weight markers in kDa are indicated on the left.

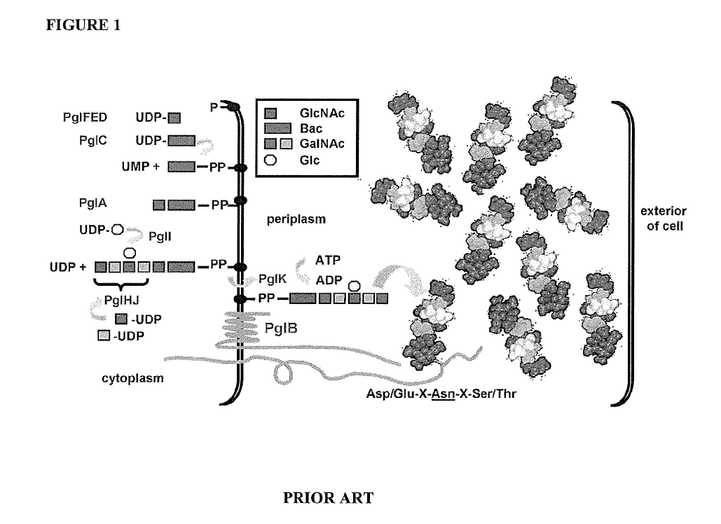

### DESCRIPTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

The term “nucleic acid sequence” (or nucleic acid molecule) refers to a DNA or RNA molecule in single or double stranded form, particularly a DNA encoding a protein or protein fragment according to the invention.

The terms “protein” or “polypeptide” are used interchangeably and refer to molecules consisting of a chain of amino acids, without reference to a specific mode of action, size, three-dimensional structure or origin. A “fragment” or “portion” of a protein may thus still be referred to as a “protein”. A “peptide”, as used herein, refers to a short polypeptide.

The term “toxoid” as used herein, refers to a bacterial toxin whose toxicity has been weakened, suppressed or inactivated either by chemical (e.g., formalin) or heat treatment, while other properties, typically immunogenicity, are maintained. As used herein, the term toxoid encompasses a protein or peptide fragment of a weakened, suppressed or inactivated toxin.

The term “gene” means a DNA sequence comprising a region (transcribed region), which is transcribed into an RNA molecule (e.g. an mRNA) in a cell, operably linked to suitable regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites.

A “chimeric gene” (or recombinant gene) refers to any gene, which is not normally found in nature in a species, in particular a gene in which one or more parts of the nucleic acid sequence are not associated with each other in nature. For example, the promoter is not associated in nature with part or all of the transcribed region or with another regulatory region. The term “chimeric gene” is understood to include expression constructs in which a promoter or transcription regulatory sequence is operably linked to one or more coding sequences or to an antisense (reverse complement of the sense strand) or inverted repeat sequence (sense and antisense, whereby the RNA transcript forms double stranded RNA upon transcription).

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous and, where necessary to join two protein encoding regions, contiguous and in reading frame so as to produce a “chimeric protein”. A “chimeric protein” is a protein composed of various protein “domains” (or motifs) which is not found as such in nature but which are joined to form a functional protein, which displays the functionality of the joined domains. A chimeric protein may also be a fusion protein of two or more proteins occurring in nature. The term “domain” as used herein means any part(s) or domain(s) of the protein with a specific structure or function that can be transferred to another protein for providing a new hybrid protein with at least the functional characteristic of the domain.

The term “target peptide”, as used herein, refers to amino acid sequences that can target a protein to an intracellular organelle or location, such as periplasmic space, or to the extracellular space (secretion signal peptide). A nucleic acid sequence encoding a target peptide can be fused (in frame) to the nucleic acid sequence encoding the amino terminal end (N-terminal end) of the protein.

A “nucleic acid construct” or “vector” is herein understood to mean a man-made nucleic acid molecule resulting from the use of recombinant DNA technology and which can be used to deliver exogenous DNA into a host cell. Vectors can comprise further genetic elements to facilitate their use in molecular cloning, such as e.g. selectable markers, multiple cloning sites and the like (see below).

The term “GlycoTag peptide”, as used herein, refers to a peptide having two or more copies of an N-linked glycosylation sequon, derived from the putative cytoplasmic protein Cj1433c, which is naturally present in C. jejuni strain 11168. The GlycoTag peptide can be in a purified or isolated form and, optionally includes glycans attached thereto. The GlycoTag peptide can be used alone or in combination with different proteins or peptides and can be incorporated into a chimeric protein or peptide for use, for example, in the development of antigen/antibody delivery systems, the preparation of vaccines, pharmaceuticals and diagnostic tests, and research into therapies for countering Campylobacter infections.

The GlycoTag peptide can be used to display any N-glycan, including those of a eukaryotic, bacterial and/or archaeal source, or N-glycan mimic generated from enzymes from any or all of the three sources. Moreover, the GlycoTag peptide can be used to display any O-glycans, where only a threonine is required.

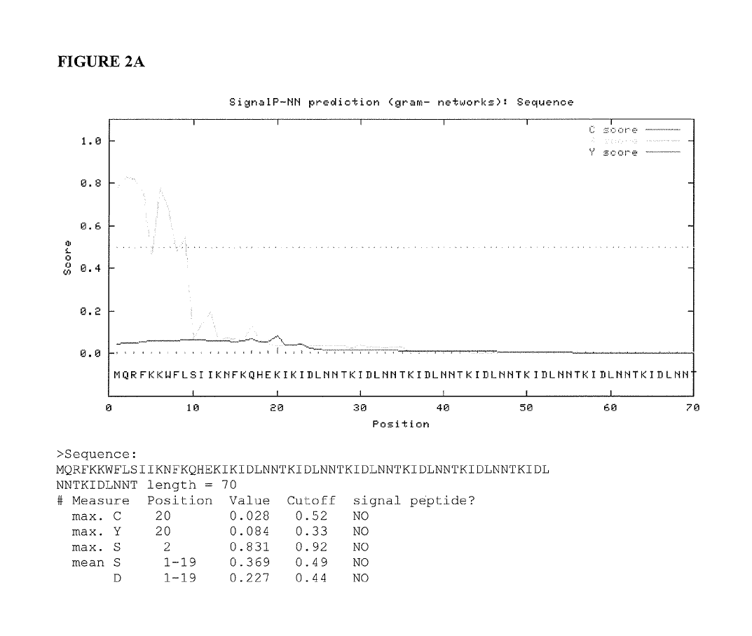

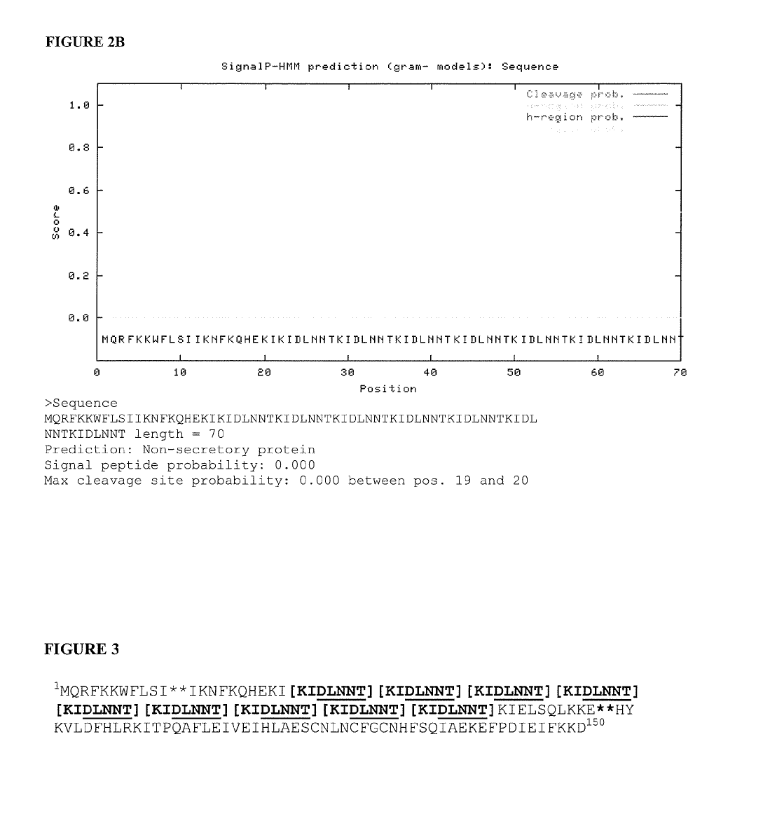

The GlycoTag peptide is derived from the N-terminal amino acid sequence of the protein Cj1433c of C. jejuni strain 11168 (FIG. 3, SEQ ID NO: 31). This segment of the protein contains nine repeats of the amino acid sequence KIDLNNT (SEQ ID NO: 1), wherein each repeat contains an N-linked glycosylation sequon (DLNNT). Bioinformatic analyses (FIGS. 2A and 2B) have shown that the Cj1433c protein containing the GlycoTag peptide is not secreted into the bacterial periplasmic space and is thus lacking one of the prerequisites for bacterial N-linked glycosylation in the native host. Moreover, use of the TMpred (“Prediction of Transmembrane Regions and Orientation”) program (http://www.ch.embnet.org/software/TMPRED_form.html) and the TMHMM program for prediction of transmembrane helices in proteins (http://www.cbs.dtu.dk/services/TMHMM-2.0/) did not predict the presence of transmembrane domains in Cj1433c. It was thus unexpected that the Cj1433c protein would contain a sequence that is highly susceptible to glycosylation. In addition to these bioinformatics analyses, a whole glycoproteome analysis performed by Scott et al. (2010) could not detect the Cj1433c protein as an N-linked glycoprotein in C. jejuni (Scott N E et al., 2010: Simultaneous glycan-peptide characterization using hydrophilic interaction chromatography and parallel fragmentation by CID, HCD and ETD-MS applied to the N-linked glycoproteome of Campylobacter jejuni. Mol Cell Proteomics, published on Apr. 1, 2010 as Manuscript M000031-MCP201. Without wishing to be bound by theory, it is clear to one of skill in the art that these analyses show that the successful use of the GlycoTag peptide as an N-glycan acceptor peptide would be unlikely. Surprisingly, the present inventors have demonstrated that is not the case. In one embodiment, to allow for N-linked glycosylation in various host systems, the GlycoTag peptide can be modified to include a signal peptide that allows for export from the host cell. In one embodiment, the signal peptide allows for export to the periplasm of bacterial cells. In one embodiment, the GlycoTag peptide is fused to a pectate lyaseB leader sequence.

In one embodiment, the GlycoTag peptide comprises between 3 and 30 repeats of the amino acid sequence set forth in SEQ ID NO: 1.

In another embodiment, the GlycoTag peptide comprises between 3 and 30 repeats of the amino acid sequence set forth in SEQ ID NO: 1, wherein the repeats are separated from one another by up to 6 amino acids.

In another embodiment, the GlycoTag peptide comprises between 3 and 30 repeats of the amino acid sequence set forth in SEQ ID NO: 1, wherein the repeats are contiguous.

In another embodiment, the GlycoTag peptide comprises at least 9 repeats of the amino acid sequence set forth in SEQ ID NO: 1, wherein the repeats are separated from one another by up to 6 amino acids.

In another embodiment, the GlycoTag peptide comprises at least 9 repeats of the amino acid sequence set forth in SEQ ID NO: 1, wherein the repeats are contiguous.

In another embodiment, the GlycoTag peptide comprises about nine repeats of the amino acid sequence set forth in SEQ ID NO: 1. the nine repeats of SEQ ID NO: 1 are contiguous. In another embodiment, the nine repeats of SEQ ID NO: 1 are separated from one another by up to 6 amino acids.

It has now been determined that the GlycoTag peptide comprising about nine N-linked glycosylation sequons expose the full, or nearly full, complement of glycan structures, thereby effectively exposing a large number of glycan moieties within a single protein or peptide. Glycosylation of a GlycoTag peptide comprising 9 repeats of the amino acid sequence set forth in SEQ ID NO: 1 can generate a mixture of glycosylated peptides each containing from 0-9 glycans (as shown using SDS-PAGE analysis).

In one example, different glycan structures can be added to the GlycoTag peptide, For example, glycosylation can be achieved using different glycosylation pathways in E. coli to add different glycans onto the glycosylations sites. In a specific example, the use of inducible promoters associated with each gene cluster (for the different glycosylation pathways) so that none allowed to fully glycosylate the GlycoTag.

Preparation of the GlycoTag Peptide

As would be readily appreciated by a worker skilled in the art, the GlycoTag peptide and proteins comprising a GlycoTag peptide can be prepared using a variety of well known methods.

In one embodiment, the GlycoTag peptide is synthesized, in whole or in part, using solid-state peptide synthesis. Alternatively, the GlycoTag peptide is prepared, in whole or in part, from isolated peptides obtained from natural sources.

In one embodiment, the GlycoTag peptide is prepared by recombinant expression of a vector comprising a nucleic acid sequence encoding the GycoTag peptide, in a host cell that optionally comprises N-linked glycosylation machinery, using techniques known in the art. In one embodiment, the host cell is E. coli that has been transformed to express the pgl machinery of C. jejuni (Wacker M et al., 2002: N-linked glycosylation in Campylobacter jejuni and its functional transfer into E. coli. Science, 298 (5599):1790-3). The construction of chimeric genes and vectors for, preferably stable, introduction of GlycoTag peptide encoding nucleic acid sequences into host cells is generally known in the art. To generate a chimeric gene the nucleic acid sequence encoding a GlycoTag peptide is operably linked to a promoter sequence, suitable for expression in the host cells, using standard molecular biology techniques. The promoter sequence may already be present in a vector so that the GlycoTag peptide nucleic sequence is simply inserted into the vector downstream of the promoter sequence. The vector is then used to transform the host cells and the chimeric gene is inserted in the nuclear genome or into a plasmid and expressed there using a suitable promoter. In one embodiment a chimeric gene comprises a suitable promoter for expression in microbial cells (e.g., bacteria), operably linked thereto a nucleic acid sequence encoding a GlycoTag peptide or fusion protein as described herein.

In one embodiment, the GlycoTag peptide is fused to an affinity tag to facilitate purification by affinity chromatography. In one embodiment, the affinity tag is fused to the N-terminus of the GlycoTag peptide. In one embodiment, the affinity tag is fused to the C-terminus of the GlycoTag peptide. Of course, as one of skill in the art will appreciate, a wide variety of different affinity tags can be used, depending on the type of affinity purification contemplated. Specific examples of affinity tags that can be used to assist in purification of the GlycoTag peptide include, but are not limited to, epitope tags, chromatography tags, and, more specifically, a hexa-histidine tag (sometime referred to as a His-tag).

Methods and Systems of Using the GlycoTag Peptide

The GlycoTag peptide can be used in a wide variety of different applications. In one embodiment, the GlycoTag peptide can be used as an adjuvant in vaccine preparation. Of course, as one of skill in the art will appreciate, the GlycoTag peptide can be engineered to carry different N-glycans depending on the intended application.

In one embodiment, the GlycoTag peptide, when glycosylated with specific glycans, can be used as a glycan carrier for vaccination against zoonotic human pathogens such as Campylobacter upsaliensis (in cats and dogs), Campylobacter coli (in pigs) and Campylobacter jejuni (in chickens). As another example, which is also not meant to be limiting, the GlycoTag peptide glycosylated with specific glycans can be used to vaccinate humans against Travelers' Diarrhea (Campylobacter jejuni) and vaccinate cows and sheep to prevent infertility and abortions (Campylobacter fetus venerealis and Campylobacter fetus fetus).

In one embodiment, the GlycoTag peptide can be co-expressed recombinantly with an acceptor protein, a peptide or an antibody using techniques known in the art. The acceptor proteins, peptides and antibodies include, but are not limited to, toxins, toxoids, periplasmic proteins, antibodies and dentritic cell targeting antibodies. In one embodiment, the protein is AcrA. In one embodiment, the toxin can be selected from toxins expressed by Bordetella pertussis, Clostridium tetani, or Corynebacterium diphtheria. Of course, many other examples are possible. This can be particularly advantageous, since co-expression of the GlycoTag peptide with an acceptor protein, peptide or antibody can serve as the basis for the preparation of a range of useful products, including vaccines against Cambylobacter or other bacteria and vaccines having improved efficacy. In one example, which is not meant to be limiting, the GlycoTag peptide, when glycosylated with specific glycans and co-expressed with other proteins and peptide, can be used as a glycan carrier for vaccination against zoonotic human pathogens such as Campylobacter upsaliensis (in cats and dogs), Campylobacter coli (in pigs) and Campylobacter jejuni (in chickens). As another example, which is also not meant to be limiting, co-expression of the GlycoTag peptide glycosylated with specific glycans with other proteins and peptides can be used to prepare vaccines against Travelers' Diarrhea (Campylobacter jejuni) and against Campylobacter fetus venerealis and Campylobacter fetus fetus.

In one embodiment of the present invention, the GlycoTag can be fused to an acceptor protein, a peptide or an antibody in order to direct or target the fusion to the periplasm of E. coli or of another expression organism. The multivalency of the GlycoTag has the advantage of attaching various glycan structures in multiple copies to a protein or peptide carrier in high density without having to engineer additional N-glycan acceptor sequences into target proteins. This can be particularly advantageous since it can allow for the recombinant expression of proteins and peptides that can display multiple N-glycan structures. As one of skill in the art will appreciate, a wide variety of carrier peptides and proteins are suitable for fusion with the GlycoTag. These include, but are not limited to, toxins, toxoids, periplasmic proteins, antibodies and dentritic cell targeting antibodies. In one embodiment, the protein is AcrA. In one embodiment, the toxin can be selected from toxins expressed by Bordetella pertussis, Clostridium tetani, or Corynebacterium diphtheria. Of course, many other examples are possible. This can be particularly advantageous, since the GlycoTag fused to a carrier protein can serve as the basis for a range of useful products, including vaccines against Cambylobacter and vaccines having improved efficacy. The GlycoTag peptide can even be used in adjuvant-free compositions. In one example, which is not meant to be limiting, the GlycoTag, when glycosylated with specific glycans, can be used as a glycan carrier for vaccination against zoonotic human pathogens such as Campylobacter upsaliensis (in cats and dogs), Campylobacter coli (in pigs) and Campylobacter jejuni (in chickens). As another example, which is also not meant to be limiting, GlycoTag glycosylated with specific glycans can be used to vaccinate humans against Travelers' Diarrhea (Campylobacter jejuni) and vaccinate cows and sheep to prevent infertility and abortions (Campylobacter fetus venerealis and Campylobacter fetus fetus). As would be readily appreciated, the GlycoTag peptide can be used in the preparation of a vaccine for any pathogen immunogenic carbohydrate epitopes. In another non-limiting example, a suitably glycosylated GlycoTag peptide can be used in the manufacture of a vaccine against the oral pathogen, C. rectus.

As one of skill in the art will appreciate, there are many different ways in which the GlycoTag can be fused to a protein or peptide. The GlycoTag peptide can be fused directly or via a linker to a protein capable of being targeted to the periplasm of a selected expression vector organism such as E. coli that, in the presence of genes encoding protein glycosylation machinery, results in N-glycosylation of the respective sites on the GlycoTag.

In one embodiment, a GlycoTag fusion peptide or protein, or chimeric protein, is prepared from expression of a chimeric gene including a coding sequence for the GlycoTag peptide operably linked to a coding sequence for a second peptide or protein. Accordingly, also provided is a vector comprising such a chimeric gene and appropriate sequences for expression of the chimeric gene in a host organism.

In one embodiment, a fusion between the GlycoTag peptide and a protein can include an affinity tag to facilitate purification by affinity chromatography. Of course, as one of skill in the art will appreciate, a wide variety of different affinity tags can be used, depending on the type of affinity purification contemplated. Moreover, depending on the nature of the fusion product, the affinity tag can be placed at the N-terminus or at the C-terminus.

In one embodiment, the GlycoTag peptide can be modified to attach any lipid-linked saccharide expressed in the presence of the GlycoTag fusion protein.

In another embodiment, the GlycoTag peptide is used in a diagnostic assay method or system. In one example of this embodiment, the GlycoTag peptide is engineered to display specific glycans characteristic of an infectious bacteria. The GlycoTag peptide can then be used in an immunological assay to detect, and optionally quantitate, antibodies directed to the infectious bacteria. In the immunological method, the GlycoTag peptide is contacted with a biological sample from a subject suspected of bacterial infection and the mixture is incubated to allow binding complexes to form between the GlycoTag peptide and the antibodies present in the sample that specific for the displayed glycans. The binding complexes are then detected using standard methods, and optionally quantitated. As would be appreciated by a worker skilled in the art, the assay can be performed using a variety of well known techniques for immunological assays. In one embodiment, the GlycoTag peptide is immobilized on a solid support prior to contact with the biological sample. The solid support can be, for example, microtitre plates, polymer beads, agarose beads, paramagnetic beads, or membranes.

In accordance with another aspect there is a provided an immunoassay kit comprising glycosylated GlycoTag peptides as described above and reagents for the detection of binding complexes formed with the glycosylated GlycoTag peptides and antibodies specific for bacterial antigens corresponding with the antigenic components of the glycosylated GlycoTag peptides. The immunoassay kit can additionally include instructions for use and/or a container or other means for collecting a sample from a subject suspected of having a bacterial infection.

In order that the invention be more fully understood, the following examples are set forth. These examples are for illustrative purposes only and are not to be construed as limiting the scope of the invention in any way. Moreover, these examples are not intended to exclude equivalents and variations of the present invention, which are apparent to one skilled in the art.

### EXAMPLES

### Example 1

### Preparation of GlycoTag/AcrA Fusion Proteins

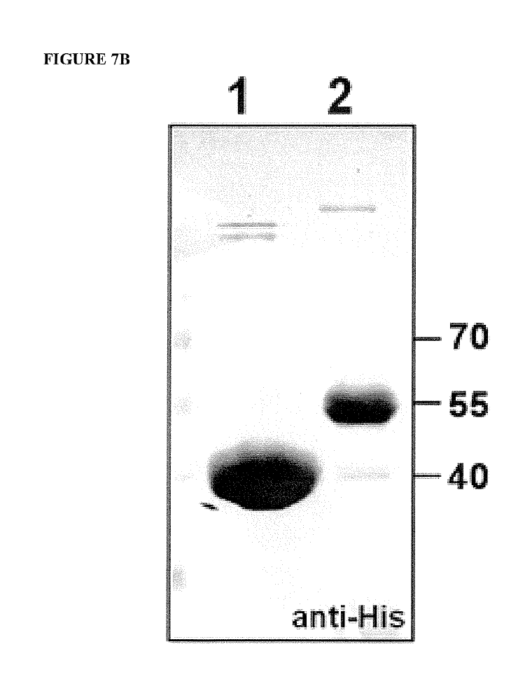

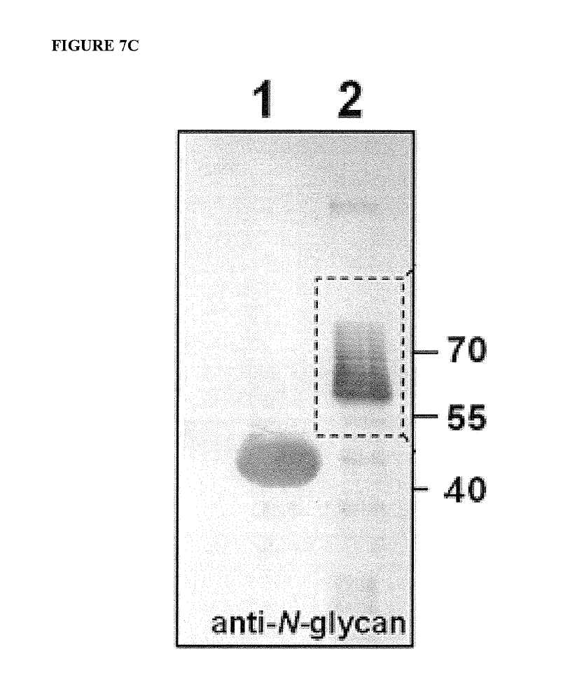

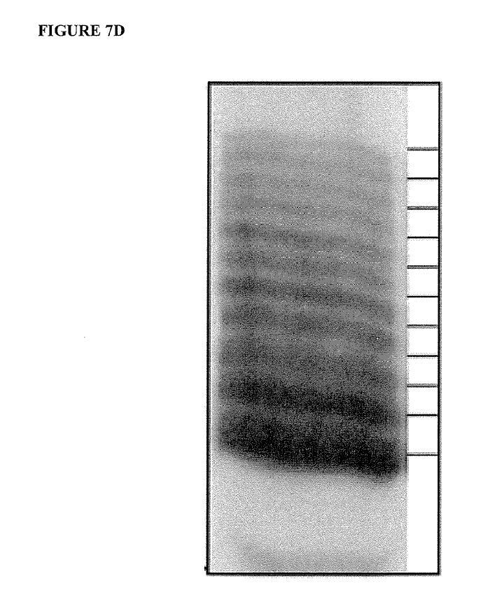

The glycoprotein AcrA of Campylobacter fetus fetus (AcrA^{Cff}), which naturally possesses two N-glycosylation sequons, was used as the protein carrier. A plasmid was constructed to express the AcrA gene of C. fetus fetus in E. coli BL21 using the pectate lyaseB (pelB) leader sequence on plasmid pET22b (Novagen) to direct the protein to the periplasmic space via the secretory (Sec) pathway. Therefore, primers AcrA^{Cff}-NcoI (5′-ATGGCCATGGATAATAAAAAATCAGCC-3′ [SEQ ID NO: 3]) and AcrA^{Cff}-XhoI (5′-TTTCTCGAGTTTGCTTCCTTTGGCATCATTTATCG-3′ [SEQ ID NO: 4]) were used to amplify nucleotides 67 to 1052 of the AcrA gene from chromosomal DNA from C. fetus fetus 82-20, while simultaneously introducing restriction sites (underlined) for NcoI and XhoI. The PCR product was inserted into plasmid pET22b, which had been digested with the same restriction enzymes. The AcrA protein is a periplasmic protein in the native host and it is also a target for N-linked glycosylation when expressed in E. coli BL21 in the presence of the C. jejuni protein glycosylation operon (after induction with 0.1 mM IPTG, FIG. 7C, lane 1).

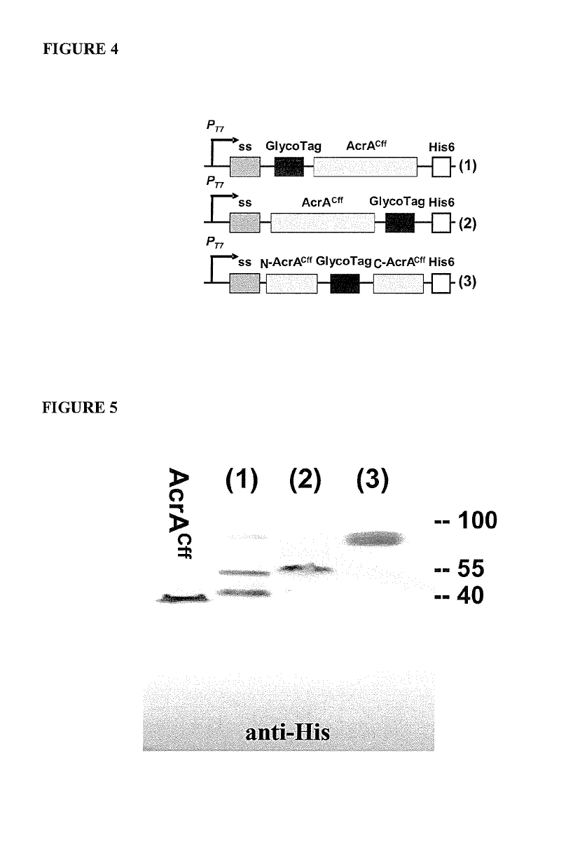

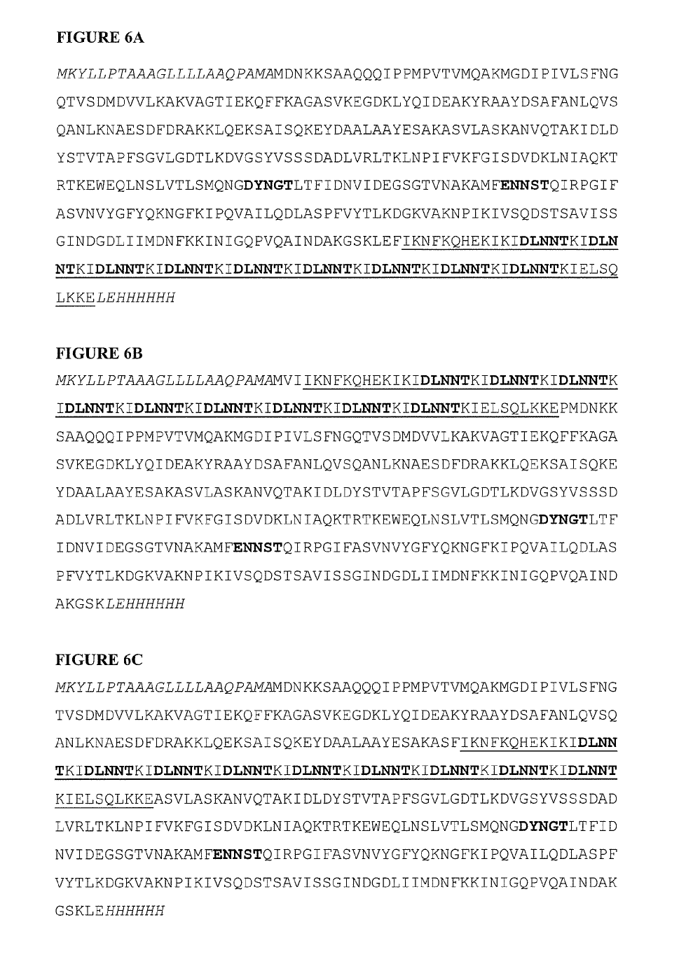

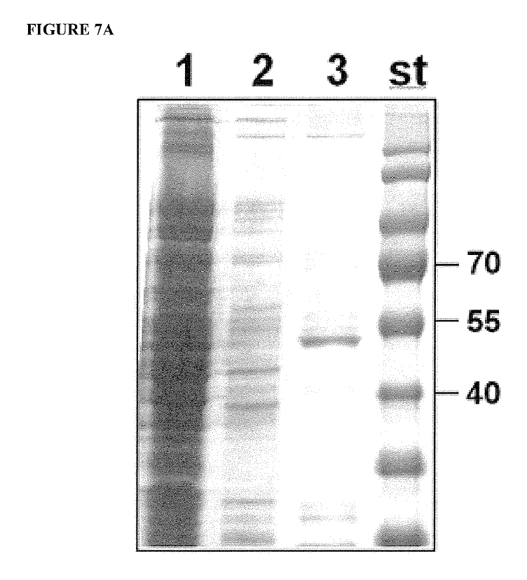

The GlycoTag peptide sequence was prepared as follows: The DNA sequence encoding the N-terminus of the Cj1433c protein [SEQ ID NO: 2], which is CTCGAGTTCATAAAAAATTTCAAGCAACATGAAAAAATTAAGATAGATCT TAATAATACAAAGATAGATCTTAATAATACAAAGATAGATCTTAATAATA CAAAGATAGATCTTAATAATACAAAGATAGATCTTAATACAAAGATA GATCTTAATAATACAAAGATAGATCTTAATAATACAAAGATAGATCTTAA TAATACAAAGATAGATCTTAATAATACAAAGATAGAATTATCGCAATTAA AAAAAGAGCTCGAG (the restriction sites for XhoI are shown in bold), was generated by PCR with gene-specific oligonucleotides (1433c-XhoI-1: 5′-AAACTCGAGTTCATAAAAAATTTCAAGC-3′ [SEQ ID NO: 5] and 1433c-XhoI-2: 5′-ATATCTCGAGCTCTTTTTTTAATTGCG-3′ [SEQ ID NO: 6]) from chromosomal DNA of the genome-sequenced strain Campylobacter jejuni 11168 (Parkhill J et al., 2000: The genome sequence of the food-borne pathogen Campylobacter jejuni reveals hyper-variable sequences. Nature, 403(6770):665-668). The digested DNA fragments were fused in frame to the N-terminus (construct 1, FIG. 4 [SEQ ID NO: 7]), to the C-terminus (construct 2, FIG. 4 [SEQ ID NO: 8]) and in between the lipoyl- and the α-hairpin domains (as described in Symmons M et al., 2009: The assembled structure of a complete tripartite bacterial multidrug efflux pump. PNAS, 106 (17): 6893-6894) of the C. fetus fetus AcrA protein (AcrA^{Cff}) at nucleotide position 457 of the AcrA^{Cff} coding sequence (construct 3, FIG. 4 [SEQ ID NO: 9]). For construct 1, the above-described PCR product was introduced via the NcoI site. For construct 2, the above-described PCR product was introduced via the XhoI site. For construct 3, the above-described PCR product was introduced via the NheI site. The correct insertion of the PCR products into the respective sites was verified by DNA sequencing. The fusion proteins (constructs 1, 2, and 3) were expressed in an E. coli BL21 expression system as follows. At an OD₆₀₀ of 0.6 (after growth at 37° C.), cultures were induced with 0.1 mM of IPTG and cells were grown for an additional 18 h at the same temperature. Cultures were cooled on ice for 15 min and cells were harvested by centrifugation (10 min, 10,000 rpm, 4° C.), washed twice in one culture volume of ice-cold PBS buffer and finally re-suspended in 1/20 of the original culture volume. Cells were disrupted by sonication (3 times 1 minute pulses) and centrifuged for 30 min at 15,000 rpm at 4° C.). His-tagged proteins were purified from the supernatants using The QiaExpressionist (Qiagen). Analyses of Ni-NTA affinity chromatography-purified proteins was followed by SDS-PAGE and Western Blot analyses with monoclonal antibodies specific against the Hexa-histidine tag (Santa Cruz Biotech Inc.). The Hexa-histidine tag-specific western blot showed that only construct 2 resulted in the overexpression of a protein with the correct size of approximately 55 kDa (FIG. 5, lane 2). The deduced amino acid sequence of the N-(construct 1), the C-(construct 2) and interdomain (construct 3) AcrA^{Cff}-GlycoTag-His fusion proteins are shown in FIGS. 6A, 6B and 6C, respectively.

### Example 2

### Expression of GlycoTag/AcrA Construct 2 in E. Coli BL21 (pgl+)

Expression in E. coli BL21 in the presence of the C. jejuni glycosylation machinery located on plasmid pACYC184 (pACYC (pgl-Cj)) (Wacker M et al., 2002: N-linked glycosylation in Campylobacter jejuni and its functional transfer into E. coli. Science, 298 (5599): 1790-1793) resulted in soluble, periplasmic expression of the C-terminal AcrA-GlycoTag-His fusion protein. The fusion protein was purified by Ni-NTA affinity chromatography and was examined by western blotting with monoclonal antibodies specific against the Hexa-histidine tag (Santa Cruz Biotechnology Inc.) and C. jejuni N-glycan-specific antiserum (hR6). FIGS. 7B and 7C show the western blots with hR6 and anti-Hexa-histidine (anti-His). A mixture of site occupancy occurs, as we observed fusion proteins with 1, 2, 3, 4, 5, 6, 7, 8, 9, 10 and 11 N-glycans attached. As shown in FIG. 7D, we could detect a chimeric glycoprotein with 11 C. jejuni heptasaccharides attached (2 natural sites of AcrA^{Cff}, 9 sites of GlycoTag peptide). This result was unpredicted because it was proposed previously that a specific protein conformation would improve glycosylation efficiency while in the current example, the N-linked glycosylation sequons (DLNNT) were separated by only two amino acids (KI) in the GlycoTag peptide.

### Example 3

### Preparation GlycoTag/Toxoid Fusion Proteins

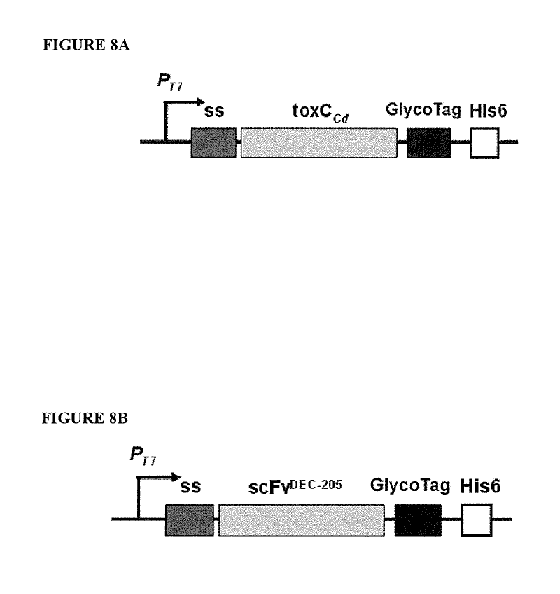

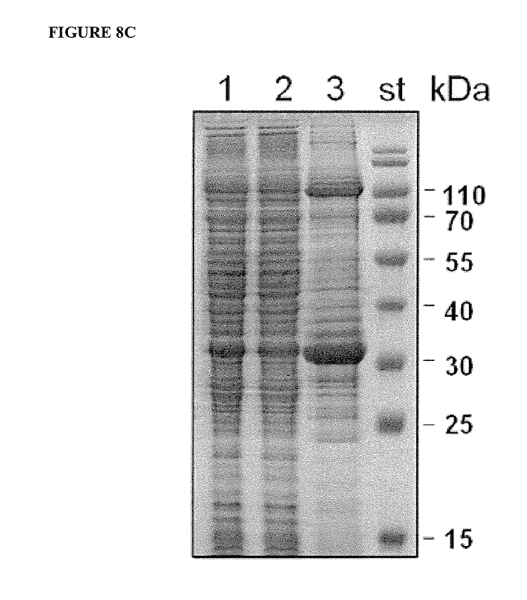

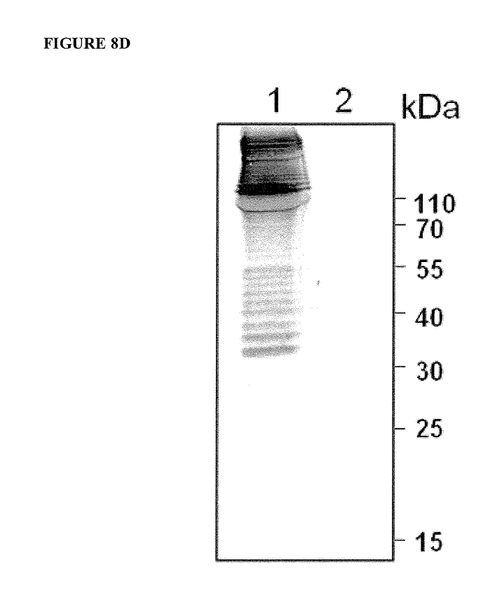

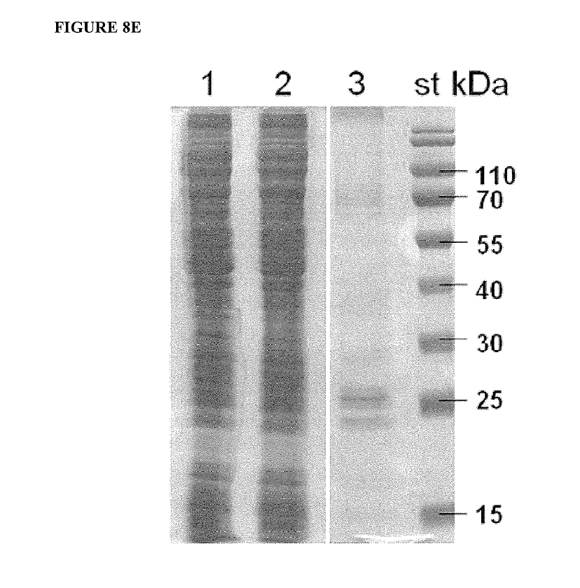

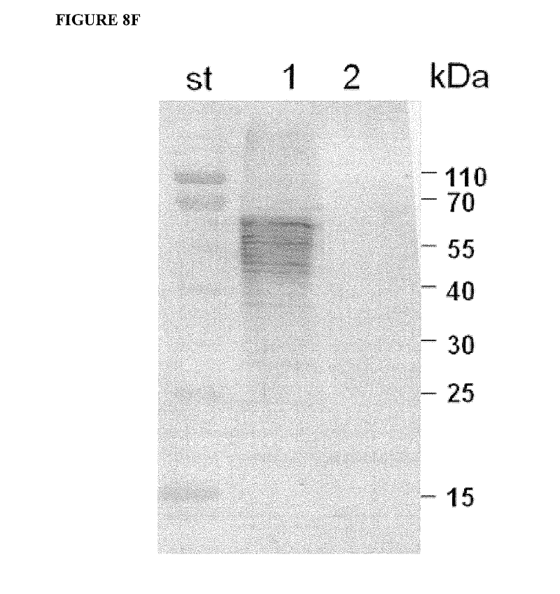

The GlycoTag sequence of SEQ ID NO: 10, prepared as described in Example 1, was fused to the N-terminus and the C-terminus of the immunostimulating toxoid gene products of Bordetella pertussis (ptxA, N-terminus, nt 1-537), Clostridium tetani (tet, C-terminus, nt 2566-3945) and Corynebacterium diphtheriae NCTC 13129 (tox_{Cd}, nt 79-654) (FIG. 8A). PCR products were generated with the following toxoid gene-specific oligonucleotides, while introducing restriction sites (NcoI, XhoI):

| | |
|---|---|
| ptxA-NcoI-1 | 5′AATATATACCATGGGTTGCACTCGGGCAATTCG-3 |
| | [SEQ ID NO: 11] |
| ptxA-XhoI-2 | 5′- ATATATCTCGAGCGTTACCCTGCGGATGTTTTC |
| | GG-3′ [SEQ ID NO: 12] |
| tet-NcoI-1 | 5′- TATATGGCCATGGCAACACCAATTCCATTTTCT |
| | TATTCTAAAAATCTGG-3 [SEQ ID NO: 13] |
| tet-XhoI-2 | 5′- TTAAACTCGAGATCATTTGTCCATCCTTCATCT |
| | GTAGGTACAAAGTACC-3′ [SEQ ID NO: 14] |
| tox-NcoI-1 | 5′- ATATATATCCATGGCTGCTGATGATGTTGTTGA |
| | TTC-3′ [SEQ ID NO: 15] |
| tox-XhoI-2 | 5′- ATATACTCGAGTCGCCTGACACGATTTCCTGCA |
| | CAGG-3′ [SEQ ID NO: 16] |

A plasmid described by Aminian et al. (2007) served as a template for the PCR reaction (Aminian et al., 2007: Expression and purification of a trivalent pertussis toxin-diphtheria toxin-tetanus toxin fusion protein in Escherichia coli. Protein Expr Purif, 51 (2): 170-178). Obtained PCR products were introduced into the plasmid expressing the N-terminal or the C-terminal GlycoTag AcrA^{Cff}-His fusion proteins. Therefore, the XhoI restriction site closer to the Hexa-histidine encoding sequence and the NcoI site closer to the pelB leader peptide encoding sequence were deleted by quick change mutagenesis using the QuikChange® Mutagenesis Kit (Stratagene) with the following oligonucleotides:

| | |
|---|---|
| QC-XhoI-Del-1 | 5′- GCAATTAAAAAAAGAGATCGAGCACCACCACC |
| | ACCACCACTGAG-3′ [SEQ ID NO: 17] |
| QC-XhoI-Del-2 | 5′- CTCAGTGGTGGTGGTGGTGGTGCTCGATCTCT |
| | TTTTTTAATTGC-3′ [SEQ ID NO: 18] |
| QC-NcoI-Del-1 | 5′- GCTGCCCAGCCGGCGATGGCGATGGTTATCAT |
| | AAAAAATTTCAAG-3′ [SEQ ID NO: 19] |
| QC-NcoI-Del-1 | 5′- CTTGAAATTTTTTATGATAACCATCGCCATCG |
| | CCGGCTGGGCAGC-3′ [SEQ ID NO: 20] |

The plasmids encoding either the N-terminal AcrA^{Cff}-GlycoTag-His or the C-terminal AcrA^{Cff}-GlycoTag-His fusion proteins served as templates.

Subsequently, the AcrA^{Cff} protein coding sequence was exchanged by the PCR-amplified toxin genes (as described above) and were inserted via NcoI-XhoI to create either the C- or the N-terminal Hexa-histidine-tagged GlycoTag-toxin fusion constructs.

Fusion protein expression, secretion into the periplasmic space of E. coli and N-glycosylation was analyzed as described above. Expression, affinity-tag purification (see above), and glycosylation in the heterologous E. coli protein glycosylation system (see above) could be demonstrated for the C-terminal pelB-toxC_{Cd}-GlycoTag-HexaHistidine construct (FIGS. 8C and 8D). Again, a mixture of site occupancy occurs, as fusion proteins were observed with 1, 2, 3, 4, 5, 6, 7, 8 and 9 N-glycans attached (FIGS. 8C and 8D). Interestingly, it appears that the N-glycan carrier peptide induces dimer formation in the fusion increasing the valency of the construct.

### Example 4

### Preparation of Two GlycoTag Peptides Fused to a Toxoid Protein

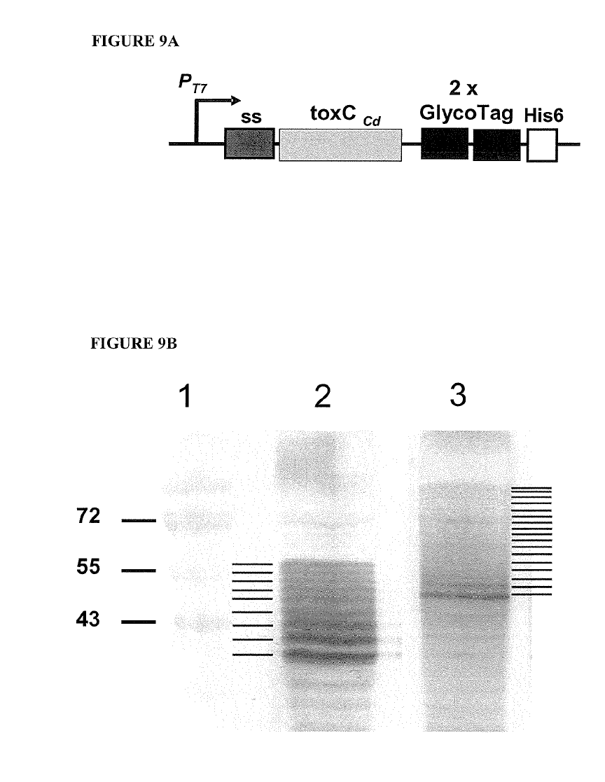

A second GlycoTag peptide of SEQ ID NO: 10 was attached to the ToxC_{Cd} fusion protein made in Example 3 to create a ToxC_{Cd}-(GlycoTag)₂ fusion protein (FIG. 9A). Therefore, a 258 bp gel-purified DNA fragment obtained after XhoI digestion of the C-terminal pelB-AcrA^{Cff}-GlycoTag-His fusion protein-encoding plasmid was inserted into the XhoI digested C-terminal pelB-ToxC_{Cd}-GlycoTag-His fusion protein-encoding plasmid. The orientation of the second GlycoTag-encoding DNA sequence was verified by DNA sequencing with the oligonucleotide hybridizing in the Hexa-histidine-encoding plasmid DNA sequence. Expression and glycosylation with the N-glycan of C. jejuni (in the presence of the C. jejuni protein glycosylation machinery-encoding plasmid) and Ni-NTA affinity chromatography was performed as described above. Analyses by western blotting with N-glycan-specific antiserum (hR6) resulted in a protein with up to 18 C. jejuni N-glycans attached (FIG. 9B). However, a mixture of site occupancy occurs, as we observed fusion proteins with 1, 2, 3, 4, 5, 6, 7, 8, 9, 10, 11, 12, 13, 14, 15, 16, 17 and 18 N-glycans attached.

### Example 5

### Preparation of GlycoTag/Antibody Fusion Proteins

The sequence encoding for the GlycoTag peptide of SEQ ID NO: 10, prepared as described in Example 1, was fused at the C-terminal and N-terminal of a single chain antibody (scFv) that recognizes the DEC-205 receptor of dendritic cells (DCs) and that can be expressed in E. coli BL21 (FIG. 8B) as described above (Wang W W et al.: 2009: A versatile bifunctional dendritic cell targeting vaccine vector. Mol Pharm, 6 (1): 158-172). Therefore, a PCR product was generated with specific

oligonucleotides (Ab-1433-XhoI,
[SEQ ID NO: 21]
5′-ATATCTCGAGGCGGCCGCAAGCTGAGGAGACTGTGACCATGACTC
C-3′,
pET-PstI
[SEQ ID NO: 22]
5′-AATTCTGCAGTAATTTTGTTTAACTTTAAGAAGG-3′

together with plasmid pWET7; Ab-1433-NcoI,

5′-ATATATATCCATGGGAGGTGGCGGATCAGAAGTGAAGC-3′ [SEQ ID NO: 23] and pET-KpnI ATATGGTACCTTAGCAGCCGGATCTCAGTGG-3′ [SEQ ID NO: 24]together with plasmid pWET5) that amplify the scFv-DEC205 gene from plasmid pWET7 (to create the C-terminal scFvDEC205-GlycoTag fusion) and from plasmid pWET5 (to create the N-terminal scFvDEC205-GlycoTag fusion) (Wang W W et al., 2009: A versatile bifunctional dendritic cell targeting vaccine vector. Mol Pharm, 6 (1): 158-172). A strategy similar to that described above for the creation of the toxoid-GlycoTag fusion proteins was applied to exchange the AcrA^{Cff} encoding DNA sequence with the scFvDEC205-encoding DNA sequence. We could express (as described above), affinity-tag purify (as described above), and glycosylate the C-terminal pelB-scFvDEC205-GlycoTag-HexaHistidine construct in the heterologous E. coli protein glycosylation system (FIGS. 8E and 8F). Again, a mixture of site occupancy occurs, as we observed fusion proteins with 1, 2, 3, 4, 5, 6, 7, 8 and 9 N-glycans attached.

The purpose of targeting the N-glycosylated GlycoTag peptide directly to dendritic cells (DC) is to reduce the amount of antigen required and also to eliminate the adjuvant requirement for immune stimulation. DCs are the antigen presenting cells of the immune system that play a critical role in innate and adaptive immune responses, especially in priming and activating T cell and B cell immunity.

### Example 6

### Immunization of Mice with GlycoTag Fusion Proteins

Six groups each with five C57BL/6 female mice were immunized with purified GlycoTag fusion proteins as set out in Table 1.

TABLE 1

| Group | Immunization material | Day 0 | Day 12 | Day 21 |
|---|---|---|---|---|
| 1 | Control | PBS | PBS | Serum |
| | | | | (Humoral) |
| 2 | Glycosylated DEC205- | 1 μg | 1 μg | Serum |
| | GlycoTag and | | | (Humoral) |
| | Anti-CD40 mAb | 25 μg | 0 | |
| 3 | Glycosylated Acr^{Cff}-GlycoTag | 1 μg | 1 μg | Serum |
| | and | | | (Humoral) |
| | Anti-CD40 mAb | 25 μg | | |
| 4 | Glycosylated ToxC_{Cd}-GlycoTag | 1 μg | 1 μg | Serum |
| | and | | | (Humoral) |
| | Anti-CD40 mAb | 25 μg | 0 | |
| 5 | Glycosylated Acr^{Cff} and | 1 μg | 1 μg | Serum |
| | Anti-CD40 mAb | 25 μg | 0 | (Humoral) |
| 6 | Unglycosylated AcrA^{Cff} and | 1 μg | 1 μg | Serum |
| | Anti-CD40 mAb | 25 μg | 0 | (Humoral) |

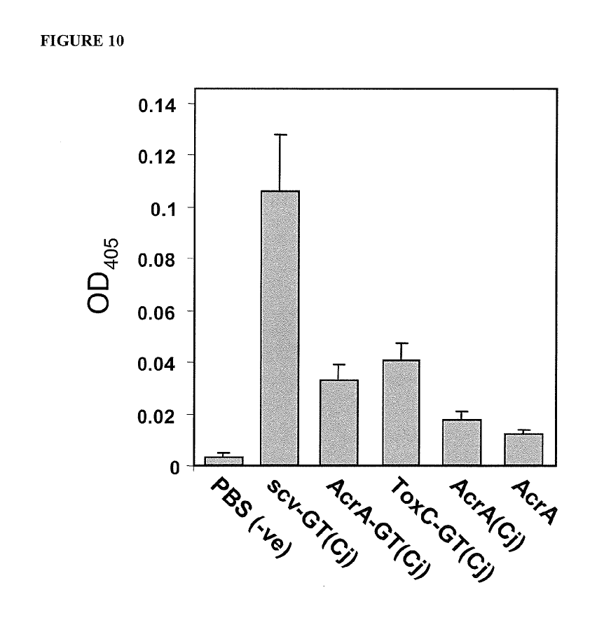

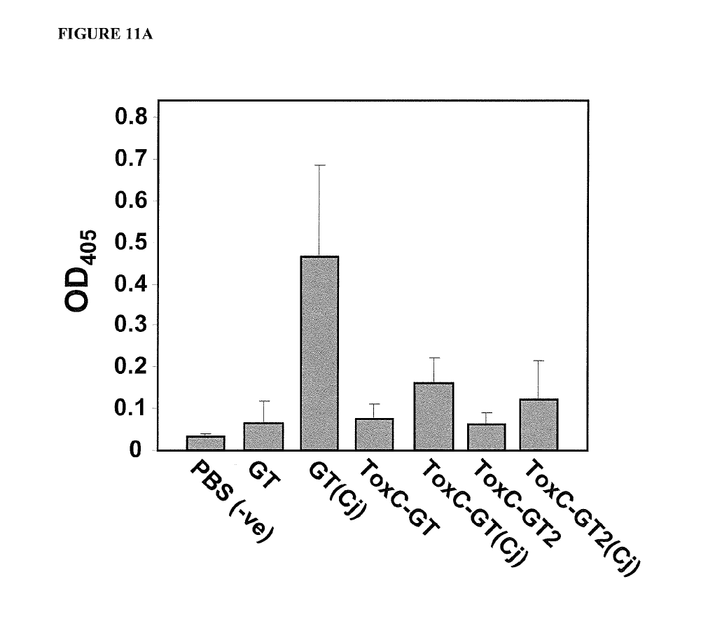

The humoral (antibody) response followed in mouse blood serum (isolated using standard procedure) of groups 1 to 6 showed that N-glycan-specific antibodies were produced in groups 2, 3, 4, and 5 in the order 2>4>3>5. No humoral response against the N-glycan was observed in groups 1 and 6 (FIG. 10).

### Example 7

### Immunization of Mice with Purified GlycoTag

Seven groups each with five C57BL/6 female mice were immunized with purified GlycoTag or purified GlycoTag fusion proteins as set out in Table 2.

TABLE 2

| | | Day 0 | Day 12 | | Day 28 | |
|---|---|---|---|---|---|---|
| | Immunization | Freund's | Freund's | | Freund's | |
| | material | complete | incomplete | Day 21 | incomplete | Day 35 |
| Gr 1 | control | PBS | PBS | Serum | PBS | Serum |
| | | | | (Humoral) | | (Humoral) |
| Gr 2 | Unglycosylated | 1 μg | 1 μg | Serum | 1 μg | Serum |
| | GlycoTag | | | (Humoral) | | (Humoral) |
| Gr 3 | Glycosylated | 2 μg = | 2 μg = | Serum | 2 μg = | Serum |
| | GlycoTag | 1 μg | 1 μg | (Humoral) | 1 μg | (Humoral) |
| | | (sugar) | (sugar) | | (sugar) | |
| Gr 4 | Unglycosylated | 30 μg | 30 μg | Serum | 30 μg | Serum |
| | ToxC_{Cd}- | | | (Humoral) | | (Humoral) |
| | GlycoTag | | | | | |
| Gr 5 | Glycosylated | 7 μg = | 7 μg = | Serum | 7 μg = | Serum |
| | ToxC_{Cd}- | 1 μg | 1 μg | (Humoral) | 1 μg | (Humoral) |
| | GlycoTag-(app. | (sugar) | (sugar) | | (sugar) | |
| | 3-9 Cj glycans) | | | | | |
| Gr 6 | Unglycosylated | 3.5 μg | 3.5 μg | Serum | 3.5 μg | Serum |
| | ToxC_{Cd}- | | | (Humoral) | | (Humoral) |
| | (GlycoTag)2 | | | | | |
| Gr 7 | Glycosylated | 3.5 μg = | 3.5 μg = | Serum | 3.5 μg = | Serum |
| | ToxC_{Cd}- | 1 μg | 1 μg | (Humoral) | 1 μg | (Humoral) |
| | (GlycoTag)2 | (sugar) | (sugar) | | (sugar) | |
| | (appr 5-18 Cj | | | | | |
| | glycans) | | | | | |

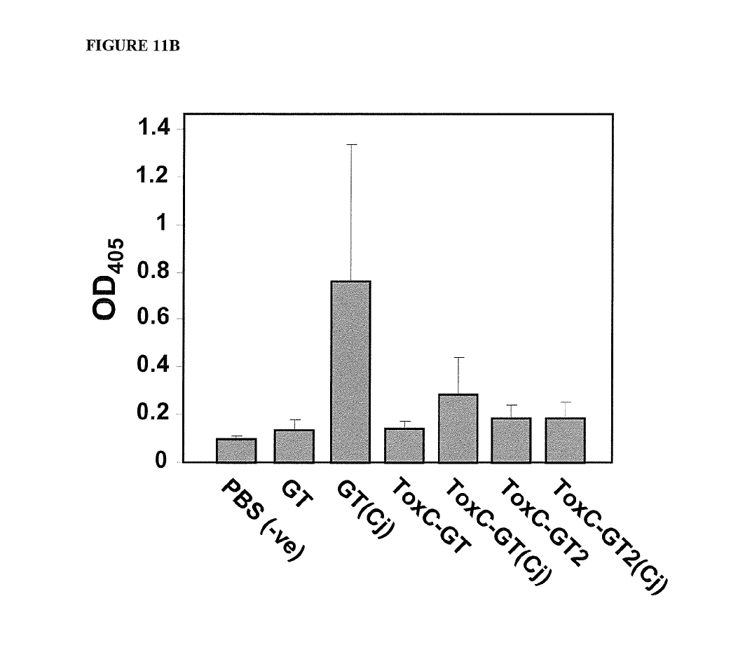

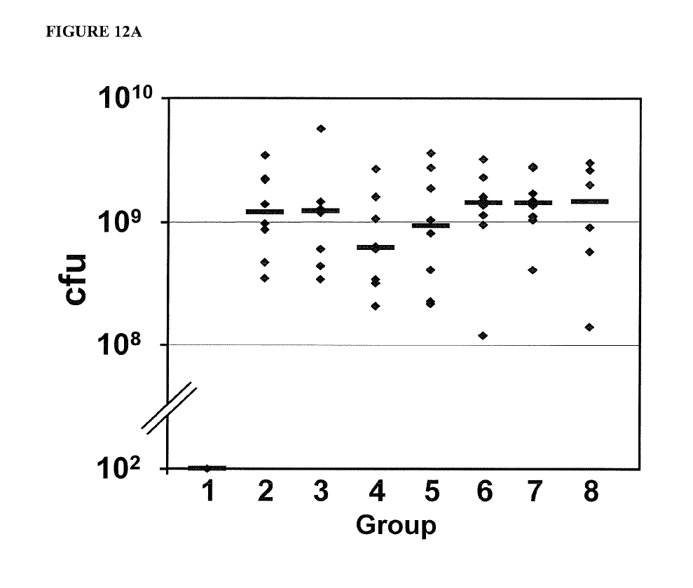

The humoral (antibody) response followed in mouse blood serum (isolated using standard procedure) of groups 1 to 6 from day 21 (FIG. 11A) and from day 35 (FIG. 11B) showed that N-glycan-specific antibodies were produced in groups 3, 5, and 7 in the order 3>5=7. No significant humoral response against the N-glycan was observed in groups 1, 2, 4 and 6.

Interestingly, these results demonstrate that use of the GlycoTag alone induced a stronger antibody response than when it was conjugated to a toxoid.

### Example 8

### Immunization of Chicken with GlycoTag Fusion Proteins

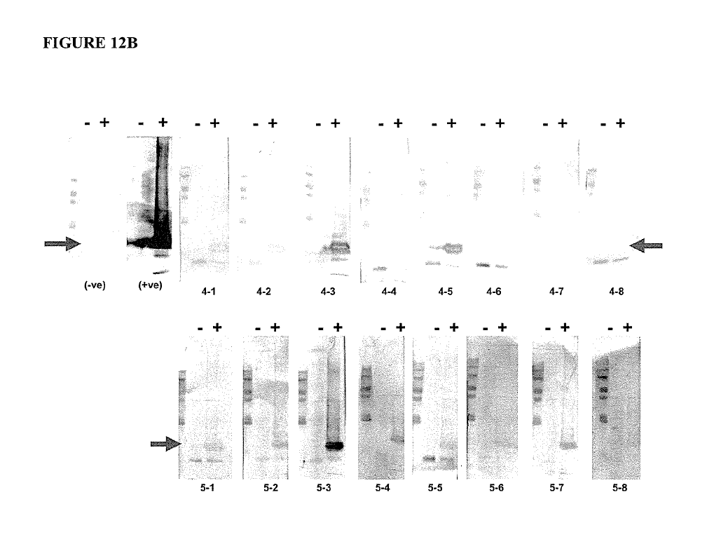

Eight groups each with eight chickens were immunized with purified GlycoTag fusion proteins as follows: At day 0, cecal swabs of 10% of all birds (5 birds were randomly selected) showed no colonization with Campylobacter when plated on selective Karamali agar plates after a 2 day incubation period under microaerobic conditions. At day 7, a pre-bleed of 500 μl was taken from each chicken followed by the 1^{st} antimicrobial treatment with 5 μg of antigen in a total volume of 300 μl for each bird (Table 3) that was administered intramuscularly (IM) by injecting 2-times 150 μl at different sites (using Freund's complete adjuvant in a 1:1 ratio with the antigen that was prepared in sterile PBS) or by oral (O) administration. At day 21, a test bleed was carried out followed by the second antimicrobial treatment with the same antigen formulation but replacing Freund's complete adjuvant with Freund's incomplete adjuvant. At day 28, chickens within groups 2-8 were gavaged (challenged) with Campylobacter jejuni (1.5×10*8 cells prepared in sterile PBS). At day 35, the chicken were euthanized, blood samples were taken from each bird (final bleed) and the colonization levels were determined by analyzing the amount of campylobacter cells in the cecum of each chicken by plating serial 10-fold serial dilutions (10*-2 to 10*-10) of the cecal contents on selective Karmali agar plates. After a 2 day incubation period under microaerobic conditions the formation of Campylobacter colonies was recorded (FIG. 12A). Reduced Campylobacter jejuni colonization levels were obtained after vaccination with ToxC-GT1, 1-9 C. jejuni (Cj)-glycans (IM) and microencapsulated (MC) ToxC-GT1, 1-9Cj-glycans (O). The humoral (antibody) response followed in blood sera (isolated using standard procedures) showed that N-glycan-specific antibodies were produced in chickens from groups 4 and 5. No humoral response against the N-glycan was observed in pooled sera of chicken from group 1 (FIG. 12B)

TABLE 3

| | | Challenge with |
|---|---|---|
| | | Campylobacter |
| | Antimicrobial treatment (5 μg of antigen) | (1.5 × 10 * 8 cfu) |
| Group 1 | PBS | no |
| Group 2 | PBS | yes |
| Group 3 | ToxC-GT1 (no glycan) (IM) | yes |
| Group 4 | ToxC-GT1, 1-9Cj-glycans (IM) | yes |
| Group 5 | Micro-encapsulated (MC) ToxC-GT1, appr. | yes |
| | 1-9 Cj-glycans (O) | |
| Group 6 | ToxC-2GlycoTags, appr 5-18 Cj glycans | yes |
| | (IM) | |
| Group 7 | ToxC-GT1, appr. 1-9 Cj-glycans (O) | yes |
| Group 8 | Microcapsules, no protein (O) | yes |

Microencapsulated antigens (MC) using chitosan (CS)-based nanoparticles were prepared by polyelectrolyte complexation of positively charged CS with the polyanion tripolyphosphate (TPP)) as described (Makhlof A., Tozuka Y., and Takeuchi H., 2011: Design and evaluation of novel pH-sensitive chitosan nanoparticles for oral insulin delivery, European Journal of Pharmaceutical Sciences, 42 (5), 445-451).

### Example 9

### Dose-Dependent Immunization of Chicken with GlycoTag Fusion Protein

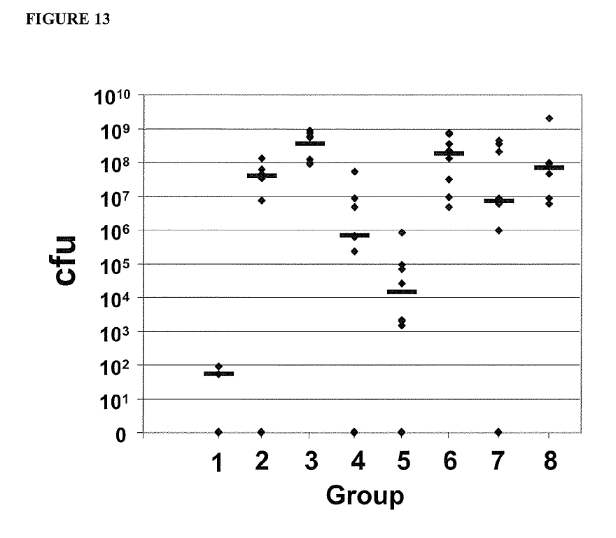

Eight groups each with eight chickens were immunized with purified GlycoTag fusion ToxC-GT1 that carries 1-9Cj-glycans by intramuscular injection as follows: 8 groups each with 8 chickens were immunized with purified GlycoTag fusion proteins as described in (Table 4). At day 0, cecal swabs of 10% of all birds (5 birds were randomly selected) showed no colonization with Campylobacter when plated on Karamali agar plates after a 2 day incubation period under microaerobic conditions. At day 7, a pre-bleed of 500 μl was taken from each chicken followed by the 1^{st} antimicrobial treatment of antigen in a total volume of 300 μl for each bird that was administered intramuscularly (IM) by injecting 2-times 150 μl at different sites (using Freund's complete adjuvant in a 1:1 ration with the antigen prepared in sterile PBS) At day 21, a test bleed was carried out followed by the second antimicrobial treatment with the same antigen formulation but replacing Freund's complete adjuvant with Freund's incomplete adjuvant. At day 28, chickens within groups 2-8 were gavaged (challenged) with Campylobacter jejuni wild-type cells (10*2 or 10*6). At day 35, the chicken were euthanized, blood samples were taken from each bird (final bleed) and the colonization levels were determined by analyzing the amount of campylobacter cells in the cecum of each chicken by plating serial 10-fold serial dilutions (10*-2 to 10*-10) of the cecal contents on selective Karmali agar plates. After a 2 day incubation period under microaerobic conditions the formation of Campylobacter colonies was recorded (FIG. 13).

TABLE 4

| | | Amount of | Challenge with |
|---|---|---|---|
| | | antigen | Campylobacter jejuni |
| | Antimicrobial treatment | (μg) | cells (cfu) |
| Group 1 | PBS | — | — |
| Group 2 | PBS | — | 10 * 2 |
| Group 3 | PBS | — | 10 * 6 |
| Group 4 | ToxC-GT1, 1-9 Cj-glycans | 5 | 10 * 2 |
| | (IM) | | |
| Group 5 | ToxC-GT1, 1-9 Cj-glycans | 100 | 10 * 2 |
| | (IM) | | |
| Group 6 | ToxC-GT1, 1-9 Cj-glycans | 5 | 10 * 6 |
| | (IM) | | |
| Group 7 | ToxC-GT1, 1-9 Cj-glycans | 100 | 10 * 6 |
| | (IM) | | |
| Group 8 | ToxC-GT1 (no glycan) | 100 | 10 * 6 |
| | (IM) | | |

Reduced Campylobacter colonization levels were obtained after vaccination with ToxC-GT1, 1-9Cj-glycans (IM) in a Campylobacter challenge dose dependent manner).

### Example 10

### Preparation of Purified MalE-GlycoTag Fusion Protein

A PCR fragment containing the GlycoTag peptide-encoding DNA (SEQ ID NO: 10) was amplified with oligonucleotides CS492 (5′-AAAATAAATATAGGACAACCGGTTCAGG-3′ [SEQ ID NO: 25]) and CS 491 (5′-TATCTCGAGTCACTCTTTTTTTAATTGCG-3′ [SEQ ID NO: 26]) using the plasmid AcrA^{Cff}-GlycoTag-His as the template, with the XhoI site deleted using the Quick Change Mutagenesis Kit (Stratagene). The PCR product was digested with XhoI, and ligated with SalI-digested p2x plasmid (New England Biotech). The correct orientation of the insert was confirmed by sequencing of the products with CS491.

Transcription of the MalE-GlycoTag fusion protein from the IPTG-inducible ptac promoter located on plasmid pMAL-p2X (New England Biolabs) in E. coli results in periplasmic expression of a MalE-GlycoTag fusion protein (SEQ ID NO: 27).

After over-expression in E. coli, the MalE-GlycoTag fusion protein can be purified using a combination of i) amylose affinity chromatography; ii) proteolytic cleavage of the MalE-GlycoTag fusion protein (e.g., with factor Xa); and/or iii) size exclusion chromatography. In all cases, the GlycoTag peptide is obtained in pure form.

### Example 11

### Preparation of GlycoTag Using a MalE-GlycoTag-His Fusion Protein

A PCR fragment containing the GlycoTag peptide-encoding DNA (SEQ ID NO: 10) was amplified with oligonucleotides CS492 (5′-AAAATAAATATAGGACAACCGGTTCAGG-3′ [SEQ ID NO: 28]) and CS 494 (5′-AATTTAAGCTTTGTTAGCAGCCGGATCTCAGTGG-3′ [SEQ ID NO: 29]) using plasmid AcrA^{Cff}-GT-His-(XhoI deleted by quick change mutagenesis) as template. The PCR product was digested with XhoI and HindIII and ligated with SalI-HindIII digested plasmid pMAL-p2X.

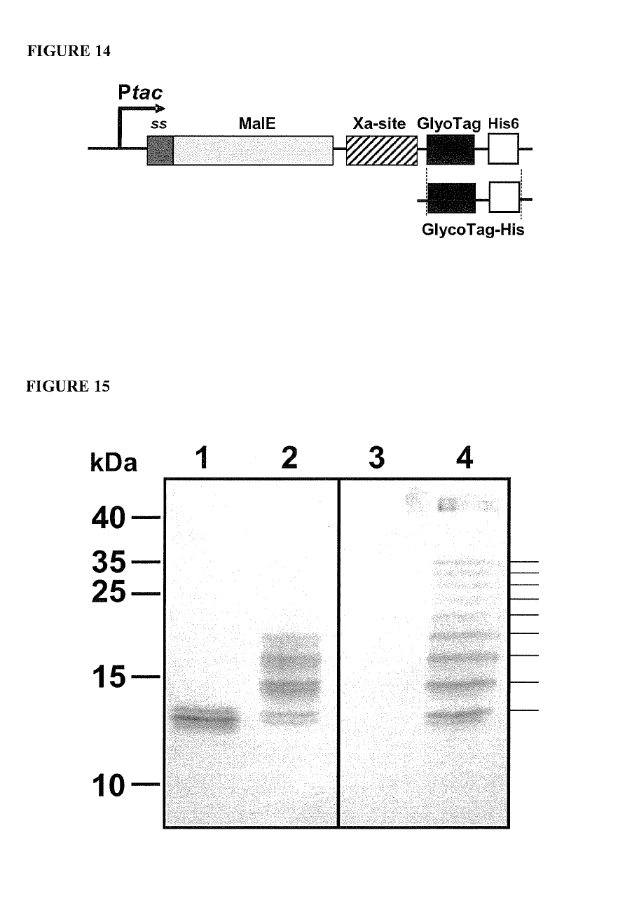

Transcription of the MalE-GlycoTag-His fusion protein from the IPTG-inducible ptac promoter located on plasmid pMAL-p2x in E. coli results in periplasmic expression of a MalE-GlycoTag-His fusion protein (SEQ ID NO: 30, FIG. 14)

After overexpression in E. coli in the presence or the absence of the Campylobacter jejuni glycosylation operon, the MalE-GlycoTag fusion protein can be purified using a combination of i) amylose affinity chromatography; ii) proteolytic cleavage of the MalE-GlycoTag fusion protein (e.g., with factor Xa, FIG. 15); and/or iii) size exclusion chromatography, Ni-NTA-affinity chromatography, or electro-elution after separation on a gel. In all cases, the GlycoTag peptide is obtained in pure form.

### Example 12

### GlycoTag as Vaccine

The GlycoTag peptide can be used as an oligosaccharide carrier peptide in immunogenicity studies against the attached oligosaccharide. The glycosylated GlycoTag can be fused to an immuno-stimulating protein (toxin) that will eliminate the need for an additional adjuvant. The glycosylated GlycoTag-peptide can be administered in combination with the preferred/standardized adjuvant for various species/applications. If necessary, purely glycosylated GlycoTag peptide can be obtained after chemical or proteolytic digestion of multiples of the glycosylated GlycoTag that is present as a mixture of GlycoTag peptide with none or 1, 2, 3, 4, 5, 6, 7, 8, or 9 sugars attached (as seen in FIG. 7D).

The glycosylation of the GlycoTag peptide yields protection from chemical or proteolytic cleavage. The oligosaccharide that is attached to the amino acid repeats (SEQ ID NO: 1) prevents cleavage by the protease trypsin. This has been confirmed by observing that non-glycosylated or incompletely glycosylated GlycoTag peptide were cleaved, whereas the glycosylated portion of the GlycoTag peptide remained intact, following exposure to protease (results not shown).

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A peptide comprising at least two repeats of the amino acid sequence as set forth in SEQ ID NO: 1.

2. The peptide of claim 1, wherein the repeats of the amino acid sequence as set forth in SEQ ID NO: 1 are contiguous or separated by no more than 6 amino acids.

3. The peptide of claim 1 comprising nine repeats of the amino acid sequence as set forth in SEQ ID NO: 1.

4. The peptide of claim 3, wherein the repeats of the amino acid sequence as set forth in SEQ ID NO: 1 are contiguous or separated by no more than 6 amino acids.

5. The peptide of claim 1, wherein the peptide further comprises a signal peptide that allows for periplasmic secretion.

6. The peptide of claim 1, wherein the peptide is glycosylated at at least one of the repeats of the amino acid as set forth in SEQ ID NO: 1.

7. A chimeric protein comprising at least one peptide as claimed claim 1 and a second peptide or a protein.

8. The chimeric protein of claim 7, wherein the second peptide or protein comprises a peptide or protein derived from, or native to, a Campylobacter, or is AcrA, a toxin, toxoid, an antibody or a periplasmic protein.

9. (canceled)

10. The chimeric protein of claim 8, wherein the toxin or toxoid is derived from Bordetella pertussis, Clostridium tetani, or Corynebacterium diphtheriae.

11. The chimeric protein of claim 8, wherein the antibody targets a dendritic cell.

12. A method for producing a chimeric protein comprising multiple glycosylation sites, comprising:
engineering a chimeric gene comprising a nucleic acid sequence encoding a peptide according to claim 1, operably linked, directly or via a linker polynucleotide, to a nucleic acid sequence encoding a second peptide or a protein; and
expressing the chimeric gene in a host.

13. The method of claim 12, wherein the second peptide or protein is AcrA, a toxin, a toxoid, an antibody or a periplasmic protein, or comprises an antibody and the chimeric protein is for use in an adjuvant-free composition for treating a Campylobacter infection.

14. (canceled)

15. The method of claim 12, wherein the second protein or peptide is derived from, or native to, a Campylobacter.

16. The method of claim 13, wherein the toxin or toxoid is derived from Bordetella pertussis, Clostridium tetani, or Corynebacterium diphtheriae.

17. The method of claim 13, wherein the antibody targets a dendritic cell.

18. (canceled)

19. The method of claim 12, further comprising glycosylating the chimeric protein with a plurality of N-linked glycans.

20.-32. (canceled)

33. A vaccine comprising one or more peptides according to claim 6 or a chimeric protein comprising the one or more peptides and a second peptide or a protein.

34. A method for vaccination of a subject employing a vaccine according to claim 33, wherein the vaccine is for a zoonotic human pathogen, Travelers' Diarrhea caused by Campylobacter jejuni, or Campylobacter coli, or periodontal disease caused by Campylobacter rectus.

35. The method according to claim 34, wherein the zoonotic human pathogen is Campylobacter upsaliensis in cats and dogs, Campylobacter coli in pigs or Campylobacter jejuni in chickens.

36. A method for vaccination of an animal employing a vaccine according to claim 33, wherein the vaccine is for vaccination of cows or sheep to prevent infertility and abortions caused by Campylobacter fetus venerealis or Campylobacter fetus fetus.

37.-41. (canceled)

42. An immunoassay kit for diagnosing a bacterial infection in a subject, the kit comprising:
(a) one or more peptides according to claim 6, wherein each peptide comprises at least one glycan that is a bacterial antigen; and
(b) instructions for contacting the one or more peptides with a biological sample from the subject and monitoring for formation of binding complexes formed by binding of the one or more peptides by antibodies in the sample that are specific for bacterial antigens.
